(19) United States
(12) Patent Application Publication
(10) Pub. No.: US 2025/0281154 A1
(43) Pub. Date: Sep. 11, 2025

(54) SYSTEMS AND METHODS FOR AUTOMATED FEATURE MEASUREMENT

(71) Applicant: Clarius Mobile Health Corp., Vancouver (CA)

(72) Inventors: Nishant Uniyal, Vancouver (CA); Nathan Woudenberg, Vancouver (CA); Wasiu Raimi, Ottawa (CA)

(21) Appl. No.: 19/071,256

(22) Filed: Mar. 5, 2025

Related U.S. Application Data
(60) Provisional application No. 63/562,227, filed on Mar. 6, 2024.

(51) Int. Cl. A61B 8/08; A61B 8/00; G06T 7/00; G06T 7/62; G06T 7/73; G06V 10/26; G06V 10/44; G06V 10/776; G06V 40/10

(52) U.S. Cl. CPC A61B 8/0866; A61B 8/463; G06T 7/0012; G06T 7/62; G06T 7/73; G06V 10/26; G06V 10/44; G06V 10/776; G06V 40/10; G06T 2207/10132; G06T 2207/30004; G06V 2201/03

(57) ABSTRACT

A method for automatically predicting, validating and measuring one or more biometric measurements on an ultrasound image comprising deploying an AI model to execute on a computing device communicably connected to the ultrasound scanner, processing, using the AI model, a new ultrasound image to identify a feature, in whole or part, and to select one or more definable values of the feature on the ultrasound image (an AI model output), calculate, using the AI model output, a validity score for the one or more definable values based on a threshold; accepting each of the one or more definable values which meet or exceed the threshold and rejecting the one or more definable values which are below the threshold; employ the one or more definable values which meet or exceed the threshold to calculate a measurement of the feature.

### CROSS-REFERENCE TO RELATED APPLICATION

This application claims the benefit of U.S. Provisional Patent Application No. 63/562,227 entitled “SYSTEMS AND METHODS FOR AUTOMATED FEATURE MEASUREMENT” filed on Mar. 6, 2024, is incorporated by reference it its entirety in this disclosure.

### FIELD OF THE INVENTION

The present disclosure relates generally to ultrasound imaging, and in particular, systems and methods for measuring features, particularly fetal features, during ultrasound imaging

### BACKGROUND OF THE INVENTION

Ultrasound imaging plays a pivotal role in monitoring fetal development and ensuring the well-being of both the mother and the unborn child during pregnancy, by providing valuable insights into various aspects of fetal growth and anatomy. Certain fetal biometric measurements, such as crown rump length (CRL), biparietal diameter (BPD), head circumference (HC), femur length (FL), and abdominal circumference (AC), serve as crucial indicators of gestational age, fetal size, and overall development. The manual acquisition of these measurements, however, is not only time-consuming but also susceptible to inter-observer variability, potentially impacting the precision of prenatal assessments.

With the recent proliferation of advanced image processing and machine learning solutions, the ability to accurately automate the acquisition of fetal biometric measurements emerges as a solution to both these challenges. In Point-of-Care Ultrasound (POCUS) settings, where operators may not always have specialized training in obstetrical imaging, the integration of automated fetal biometric measurements becomes particularly valuable. Providing AI assistance in these environments can serve as a quality assurance tool, helping the operators capture high quality, clinically relevant obstetrical images. This is especially impactful in rural and remote settings, where specialized obstetrical sonography services may not be available, preventing the need for patients to travel to larger urban centres in order to obtain medical imaging. The accessibility and portability of ultrasound devices in POCUS settings make them integral tools for obstetricians, family doctors, midwives, and other healthcare providers who need real-time information to guide clinical decision-making during prenatal examinations.

Accurate knowledge of gestational age is the key to successful antepartum care and for successful planning of appropriate intervention or treatment. Multiple standard fetal biometric charts are available for prediction of Gestational Age (GA) from the given fetal parameters which include measurement of gestational sac, CRL, fetal BPD, HC, AC, and FL. The most frequently used parameters in the second and third trimester of pregnancy are the BPD, HC, AC and FL. These parameters are considered as the ‘gold standard’ as they collectively assess the GA to the highest degree of accuracy. The most frequently used parameter in the first trimester of pregnancy is CRL, which becomes more difficult to use in later gestation, as the fetus increases in size and view on an ultrasound image.

Fetal HC biometric measurement is an important item of fetal examination, which is generally measured in the specific cross section of fetal head (called standard plane). Obstetricians and gynecologists can estimate the gestational age and fetal weight at the 13th to 25th week of pregnancy by measuring the fetal HC, evaluate the development of the fetus, and determine the delivery mode of pregnant women. Most often, at present, the fetal HC is generally measured manually by radiologists with ellipse fitting, which is time-consuming and tedious, and may cause inter- and intra-operator error and challenges.

For all measurements, an ultrasound operator must determine the specific view which is imaged in order for the measurements to be taken and subsequently the operator must place the correct number of calipers to by manipulation on user interface screen. The issues with manual placement are increased with the use of modern, portable ultrasound medical imaging systems (POCUS or point of care ultrasound systems) which connect to off-the-shelf display computing devices such as those running iOS™ or Android™ operating systems (for example via smart phones and tablets). As compared to traditional cart-based and dedicated ultrasound systems that have keyboards, a trackball or other physical input controls, these off-the-shelf display computing devices typically receive input from users via touchscreens. While the use of these touchscreen inputs may allow for a more familiar user interface, it may be difficult to be as precise using touchscreen input versus the physical controls of traditional ultrasound systems.

One area where this lack of precision may present a challenge is performing measurements on ultrasound images such as those required to place calipers for feature measurements. Traditional manual approaches to caliper placement involve placing a first edge of the caliper on one side of the imaged structure to be measured, and then placing the second edge of the caliper on an opposing side of the imaged structure to be measured. Using a touchscreen to precisely place the edges of the calipers may be difficult since a fingertip of an ultrasound operator may typically be larger than that of the arrowhead of a cursor manipulated by manual controls (e.g., a trackball). These challenges may be even more pronounced in instances where the ultrasound operator is wearing protective gloves as they have less tactile feedback about finger placement.

Additionally, the screen size of off-the-shelf display computing devices vary greatly. In certain instances, measurements may be performed on tablet-sized computing devices with larger displays, and distances between points for caliper placement may be easily positioned. However, in certain other instances, the off-the-shelf display computing devices may also be smartphones with smaller display sizes. In these instances, a fingertip may have less pinpoint accuracy and it may be difficult to perform a measurement if the distance that is desired to be measured is small. For example, it may be difficult to place the two edges of a caliper on an ultrasound image because the two points are displayed close together on a smaller display.

Some traditional attempts at addressing these challenges include using measurement tools to automatically place calipers. However, these automatic tools rely on image analysis techniques that may not be accurate, and thus, may result in incorrect caliper placements. For example, some of these image analysis tools include contour identification techniques (e.g., an active contour model or “snakes” algorithm) that attempt to identify a structure within an ultrasound image. However, these algorithms typically require complex mathematical operations such as solving of differential equations that are computationally intensive. This may make them difficult to perform on mobile devices that have limited processing capabilities and battery power.

These and other drawbacks further increase the complexity and inaccuracy of feature measurements via ultrasound. There is thus a need for improved ultrasound systems and methods for performing a measurement of an ultrasound image displayed on a touchscreen device and, in particular, for performing measurements of one or more fetal biometric measurements in an ultrasound image. The embodiments discussed herein may address and/or ameliorate at least some of the aforementioned drawbacks identified above. The foregoing examples of the related art and limitations related thereto are intended to be illustrative and not exclusive. Other limitations of the related art will become apparent to those of skill in the art upon a reading of the specification and a study of the drawings herein.

### BRIEF DESCRIPTION OF THE DRAWINGS

Non-limiting examples of various embodiments of the present disclosure will next be described in relation to the drawings, in which:

FIG. 1 is a flowchart diagram showing steps of a method for automatically predicting, validating and measuring one or more biometric measurements on an ultrasound image displayed on a device, in accordance with at least one embodiment of the present invention;

FIG. 2 is another flowchart diagram showing steps of a method, performed by a trained AI model and workflow module, in accordance with at least one embodiment of the present invention;

FIG. 3 is another flowchart diagram showing steps of a method, performed by a trained AI model and workflow module, in accordance with at least one embodiment of the present invention;

FIG. 4 is another flowchart diagram showing the steps of a method, performed by a trained AI model and workflow module, in accordance with at least one embodiment of the present invention;

FIG. 5 is a schematic architecture of the system of the present invention as applied to an image comprising a fetal head;

FIG. 6 is an image showing various segmentation mask outputs, in accordance with at least one embodiment of the present invention;

FIG. 7 is an image showing the result of performing acts of the present methods on an example ultrasound image, in accordance with at least one embodiment of the present invention;

FIG. 8 is an image showing the result of performing acts of the present methods on an example ultrasound image, in accordance with at least one embodiment of the present invention;

FIG. 9 is an image showing the result of performing acts of the present methods on an example ultrasound image, in accordance with at least one embodiment of the present invention;

FIG. 10 is an image showing the result of performing acts of the present methods on an example ultrasound image, in accordance with at least one embodiment of the present invention;

FIG. 11 is an image showing the result of performing acts of the present methods on an example ultrasound image, in accordance with at least one embodiment of the present invention;

FIG. 12A is an image showing the result of performing acts of the present methods on an example ultrasound image, in accordance with at least one embodiment of the present invention;

FIG. 12B is an image showing the result of performing acts of the present methods on an example ultrasound image, in accordance with at least one embodiment of the present invention

FIG. 13A is an image showing the result of performing acts of the present methods on an example ultrasound image, in accordance with at least one embodiment of the present invention

FIG. 13B is an image showing the result of performing acts of the present methods on an example ultrasound image, in accordance with at least one embodiment of the present invention

FIG. 14A is an image showing the result of performing acts of the present methods on an example ultrasound image, in accordance with at least one embodiment of the present invention

FIG. 14B is an image showing the result of performing acts of the present methods on an example ultrasound image, in accordance with at least one embodiment of the present invention

FIG. 15A is an image showing the result of performing acts of the present methods on an example ultrasound image, in accordance with at least one embodiment of the present invention

FIG. 15B is an image showing the result of performing acts of the present methods on an example ultrasound image, in accordance with at least one embodiment of the present invention

FIG. 16 is an image showing the result of performing acts of the present methods on an example ultrasound image, in accordance with at least one embodiment of the present invention

FIG. 17 is an image showing the result of performing acts of the present methods on an example ultrasound image, in accordance with at least one embodiment of the present invention

FIG. 18 is a schematic diagram of the training and deployment of an AI model, according to an embodiment of the present invention;

FIG. 19 is flowchart diagram of the steps for training the AI model, according to an embodiment of the present invention;

FIG. 20 is a schematic diagram of an ultrasound imaging system, according to an embodiment of the present invention; and

FIG. 21 is a schematic diagram of a system with multiple ultrasound scanners, according to an embodiment of the present invention.

Unless otherwise specifically noted, articles depicted in the drawings are not necessarily drawn to scale.

### DETAILED DESCRIPTION OF THE INVENTION

### A. Glossary

The term “AI model” means a mathematical or statistical model that may be generated through artificial intelligence techniques such as machine learning and/or deep learning. For example, these techniques may involve inputting labeled or classified data into a neural network (e.g., a deep neural network) algorithm for training, so as to generate a model that can make predictions or decisions on new data without being explicitly programmed to do so. Different software tools (e.g., TensorFlow™, PyTorch™, Keras™, U-Net model with MobileNetV2 backbone (MNV2 U-Net)) may be used to perform machine learning processes. Within the scope of the invention, an AI model is trained so that when the AI model is deployed, a computing device identifies a feature to be measured, in whole or part and selects a definable value of the feature as described further herein. It is to be understood that the present invention is not to be limited to any one means of deploying an AI model or to any one particular AI model output whether than be feature identification and/or segmentation and/or heat map creation and/or co-ordinate output. Depending on the structure of the feature to be measured (linear vs circumferential), an AI model may be differently deployed and the output(s) likewise different, as described further herein. Notwithstanding these differences, an AI model output, in accordance with the present invention, is subjected to a validation module.

The term “back converting” also, back conversion or back convert or any of its grammatical forms, means to perform a reverse scan conversion, which is to convert an ultrasound image back to its corresponding raw ultrasound data frame, or to a raw ultrasound data frame in a standardized format. The standardized format may be defined, for example, by a fixed number of scan lines and a fixed number of samples in each scan line. Back converting may also apply to markings made on an ultrasound image, which may delineate an anatomical feature, in which case the coordinates of the markings are transformed from the coordinate system of the ultrasound image to the coordinate system of the raw ultrasound data frame. The back converting of markings and their insertion, combination or association with a raw ultrasound data frame may be considered to be an interpolation of the markings into the raw ultrasound data frame.

The term “caliper set” and “calipers” (whether singular or plural) refers generally to a pair of digital measuring lines, i.e. connected point to point, (viewable on a display screen, on an ultrasound image) or a continuous/substantially continuous line, for example, used to determine one or more circumferential dimensions of, for example, HC and AC and/or one or more one or more cross-sectional/linear (landmark) measurements of, for example, FL, BPD, CRL on an ultrasound image. Within an embodiment of the present invention, measurements are taken and saved in a workflow associated with an application (for example, the Clarius™ App) when on a live imaging screen after an ultrasound image has been frozen, as well as on a captured ultrasound image prior to an examination being completed. Measurements may be performed by automatic placement of one or more calipers on the display screen, based upon an AI model output, which is then employed by a validity module to calculate a validity score for the one or more definable values based on a threshold wherein each of the one or more definable values which meet or exceed the threshold are employed to calculate a measurement of a feature. Generally, a center of the crosshairs of the caliper correlate to the ultrasound pixel that is focused on for making an exact measurement.

The term “cartesian coordinate” or “cartesian coordinate system in a plane” is an x-y coordinate system that specifies each point uniquely by a pair of numerical coordinates, which are the signed distances to the point from two fixed perpendicular oriented lines, measured in the same unit of length. Each reference coordinate line is called a coordinate axis or just axis (plural axes) of the system, and the point where they meet is its origin, at ordered pair (0, 0). The coordinates can also be defined as the positions of the perpendicular projections of the point onto the two axes, expressed as signed distances from the origin.

The term “communications network” and “network” can include both a mobile network and data network without limiting the term's meaning, and includes the use of wireless (e.g. 2G, 3G, 4G, 5G, WiFi®, WiMAX®, Wireless USB (Universal Serial Bus), Zigbee®, Bluetooth® and satellite), and/or hard wired connections such as local, internet, ADSL (Asymmetrical Digital Subscriber Line), DSL (Digital Subscriber Line), cable modem, T1, T3, fiber-optic, dial-up modem, television cable, and may include connections to flash memory data cards and/or USB memory sticks where appropriate. A communications network could also mean dedicated connections between computing devices and electronic components, such as buses for intra-chip communications.

The term “coordinate converting” or any of its grammatical forms, refers to the conversion of data from polar coordinates to cartesian coordinates (scan conversion as define below) or cartesian coordinates to polar coordinates (back converting as defined above). Ultrasound scanners gather data in the form of polar coordinates whereas conventional display interfaces, such as for example those on multi-purpose electronic devices, comprise a rectangular grid, and this grid configuration requires the use of cartesian coordinates to enable display of images thereon.

The term “labeling” refers to an act of labeling either a piece of training data or non-training data. For example, a user may mark a feature on an ultrasound image and identify the anatomy to which the feature corresponds. The result is a labeled piece of data, such as a labeled ultrasound image. Alternatively, and by way of example, an AI model may automatically and without user intervention label one or more segmented features, within an ultrasound image.

The term “module” can refer to any component in this invention and to any or all of the features of the invention without limitation. A module may be a software, firmware or hardware module (or part thereof), and may be located or operated within, for example, in the ultrasound scanner, a display device or a server. Notwithstanding this generality, the term “validation module” can refer to a workflow, and/or software and/or application which receives one or more AI model output(s) and calculates, using the AI model output, a validity score for the one or more definable values based on a threshold. Within a preferred aspect of the present invention, a trained AI model produces at least two types of outputs: i) segmentations; and ii) landmarks. Both outputs undergo post-processing including, at least the calculation by a validity module, of a “validity” value was for both output types, based upon comparisons to a threshold. This module may be referred to herein interchangeably as “validity module” and “validity and measurement module”. For example, segmentation validity is based on how elliptical a predicted a segmentation mask of a circumferential feature appears. For example, landmark validity is based on an intensity of a landmark/landmark feature's predicted heatmap localized around an associated coordinate. In this way, validity values are used in the post-processing of the AI model predictions to filter out low-confidence landmarks using a thresholding operation (i.e., all predictions with validity scores below a certain value are considered invalid). Table 2 shows exemplary network outputs associated with target measurements along with their respective validity thresholds. As described further herein, as a consequence of a defined value meeting or exceeding a threshold in the validity module, calipers may be placed and a measurement from the point(s) of the calipers acquired. The quality control enabled by the method and system of the invention means that a defined value which is below a threshold does not trigger a measurement.

The term “multi-purpose electronic device” or “display device” or “computing “device” or “off-the-shelf display computing device” is intended to have broad meaning and includes devices with a processor communicatively operable with a screen interface, for example, such as, laptop computer, a tablet computer, a desktop computer, a smart phone, a smart watch, spectacles with a built-in display, a television, a bespoke display or any other display device that is capable of being communicably connected to an ultrasound scanner. Such a device may be communicatively operable with an ultrasound scanner and/or a cloud-based server (for example via one or more communications networks).

The term “operator” (or “user”) may (without limitation) refer to the person that is operating an ultrasound scanner (for example, a clinician, medical personnel, a sonographer trainer, a student, a vet, a sonographer/ultrasonographer and/or ultrasound technician). This list is non-exhaustive.

The term “processor” can refer to any electronic circuit or group of circuits that perform calculations, and may include, for example, single or multicore processors, multiple processors, an ASIC (Application Specific Integrated Circuit), and dedicated circuits implemented, for example, on a reconfigurable device such as an FPGA (Field Programmable Gate Array). A processor may perform the steps in the flowcharts and sequence diagrams, whether they are explicitly described as being executed by the processor or whether the execution thereby is implicit due to the steps being described as performed by the system, a device, code or a module. The processor, if comprised of multiple processors, may be located together or geographically separate from each other. The term includes virtual processors and machine instances as in cloud computing or local virtualization, which are ultimately grounded in physical processors.

The term “scan convert”, “scan conversion”, or any of its grammatical forms refers to the construction of an ultrasound media, such as a still image or a video, from lines of ultrasound scan data representing echoes of ultrasound signals. Scan conversion may involve converting beams and/or vectors of acoustic scan data which are in polar (R-theta) coordinates to cartesian (X-Y) coordinates.

The term “system” when used herein, and not otherwise qualified, refers to a system for method for automatically predicting, validating and measuring one or more biometric measurements on an ultrasound image. In various embodiments, the system may include an ultrasound scanner and a multi-purpose electronic device/display device; and/or an ultrasound scanner, multi-purpose electronic device/display device and a server. The system may include one or more applications operating on a multi-purpose electronic device/display device to which the ultrasound scanner is communicatively connected.

The term “ultrasound image frame” (or “image frame” or “ultrasound frame”) refers to a frame of either pre-scan data or post-scan conversion data that is suitable for rendering an ultrasound image on a screen or other display device.

The term “ultrasound transducer” (or “probe” or “ultrasound probe” or “transducer” or “ultrasound scanner” or “scanner”) refers to a wide variety of transducer types including but not limited to linear transducer, curved transducers, curvilinear transducers, convex transducers, microconvex transducers, and endocavity probes. In operation, an ultrasound scanner is often communicatively connected to a multi-purpose electronic device/display device to direct operations of the ultrasound scanner, optionally through one or more applications on the multi-purpose electronic device/display device (for example, via the Clarius™ App).

The term “workflow application” or “application” (for example, via the Clarius™ App) or “workflow” refers to a software tool that automates the tasks involved in the validation and feature measuring process including, but not limited to one or more of the following method steps: i) receiving one or more outputs of the trained AI model of the present invention, ii) calculating, using the AI model output, a validity score for the one or more definable values based on a threshold; iii) placing one or more caliper sets based on accepted co-ordinates and/or the degree of overlap between one or more parts of the contour and an ellipse; iv) calculating a measurement using automatically placed caliper set(s); v) calculating a gestational age based upon at least one of said measurements; and iv) conveying to an operator, one or more of the measurements and if the ultrasound image comprises a fetus, the gestational age of a fetus. In some embodiments of the invention, the workflow application guides the entire process automatically, issuing screen display notifications to users/operators as needed, with triggers to complete tasks or with specific commands, for example, after processing an ultrasound image and a resultant validity value/score for said ultrasound image is below a threshold, a prompt may be provided to select an additional or alternative image, for processing through the workflow. In some aspects of the invention, validated and quality controlled measurements only require that the workflow tool be activated once, where the workflow enables: i) activation of an AI model to create AI model outputs; ii) automatic calculation of validity values/scores as compared to a threshold using one or more AI model outputs; iii) appropriate placement of caliper set(s), based upon resultant validity values/scores meeting or exceeding a threshold; iv) automatic measurement; and v) conveyance to an operator, one or more of the measurements and if the ultrasound image comprises a fetus, the gestational age of a fetus. Conveyance to an operator may be visually on the display screen or via audio.

### B. Exemplary Embodiments

In a first broad aspect of the present disclosure, there are provided ultrasound systems, ultrasound-based methods, tools and workflows for automatically predicting one or more fetal biometric measurements on an ultrasound image comprising a fetus, in whole or part, and then automatically generating a prediction of fetal gestational age, based on the said one or more fetal biometric measurements.

In another aspect of the present disclosure, there are provided ultrasound systems, ultrasound-based methods, tools and workflows for assigning at least one validity score relating to each of one or more definable values, associated with a feature on an ultrasound image, thereby creating a degree of confidence of the one or more definable values, based on a threshold, wherein only the one or more definable values which meet or exceed the threshold are employed to calculate a measurement of the feature.

In another aspect of the present disclosure, there is provided a method for automatically predicting one or more biometric measurements on an ultrasound image which comprises acquiring an ultrasound image, from an ultrasound scanner; deploying an AI model to execute on a computing device communicably connected to the ultrasound scanner, wherein the AI model is trained so that when the AI model is deployed, the computing device identifies a feature to be measured, in whole or part and selects a definable value of the feature; processing, using the AI model, the ultrasound image to identify a feature, in whole or part, and to select one or more definable values of the feature on the ultrasound image, together forming an AI model output; calculating, using the AI model output, a validity score for the one or more definable values based on a threshold; accepting each of the one or more definable values which meet or exceed the threshold and rejecting the one or more definable values which are below the threshold; employing the one or more definable values which meet or exceed the threshold to calculate a measurement of the feature.

In another aspect of the present disclosure, there is provided a method for automatically predicting one or more biometric measurements on an ultrasound image which comprises acquiring, from an ultrasound scanner, an ultrasound image comprising a landmark feature wherein a measurement of the feature is linear, and a definable value of the feature comprises one or more co-ordinates, deploying an AI model to execute on a computing device communicably connected to the ultrasound scanner to process the ultrasound image and to generate at least one heatmap comprises a probability of finding localization of said landmark feature; and transforming at least one heatmap to normalized coordinates of the landmark feature using a Differentiable Spatial to Numerical Transform; calculating a validity score for each co-ordinate, of the normalized co-ordinates, based on the intensity of the at least one heatmap relative to x-y co-ordinates of a centre of mass of the feature, wherein when the validity score for a co-ordinate is below the threshold, the co-ordinate is rejected and wherein when the validity score for a co-ordinate is at or above the threshold, the co-ordinate is accepted; calculating the measurement using the co-ordinates which are accepted.

In another aspect of the present disclosure, there is provided a method for automatically predicting one or more biometric measurements on an ultrasound image which comprises acquiring, from an ultrasound scanner, an ultrasound image comprising a circumferential feature and a definable value of the feature comprises a predicted contour area and the method comprises deploying an AI model to execute on a computing device communicably connected to the ultrasound scanner to process the ultrasound image and to generate at least one segmentation mask of the circumferential feature thereby forming a contour in the segmentation mask; fitting an ellipse on the contour; calculating a validity score for one or more parts of the contour, based on the degree of overlap between one or more parts of the contour and the ellipse, wherein when the validity score is below the threshold, no circumference measurement is calculated and when the validity score is at or above the threshold, the contour is accepted as a high validity segmentation and the circumference of the circumferential feature is calculated.

In another aspect of the present disclosure there is provided an ultrasound imaging system for automatically predicting one or more biometric measurements on an ultrasound image comprising an ultrasound scanner configured to acquire a new ultrasound image frame; a computing device communicably connected to the ultrasound scanner and configured to process the new ultrasound image frame against a trained AI model to i) identify a feature on the ultrasound image, in whole or part, and to select one or more definable values of the feature on the ultrasound image, together forming an AI model output; ii) calculate, using the AI model output, a validity score for the one or more definable values based on a threshold; iii) accept each of the one or more definable values which meet or exceed the threshold and reject the one or more definable values which are below the threshold; iv) employ the one or more definable values which meet or exceed the threshold to calculate a measurement of the feature.

In another aspect of the present disclosure there is provided computer-readable media storing computer-readable instructions, which, when executed by a processor communicatively coupled with an ultrasound scanner, cause the processor to i) identify a feature on the ultrasound image, in whole or part, and to select one or more definable values of the feature on the ultrasound image, together forming an AI model output; ii) calculate, using the AI model output, a validity score for the one or more definable values based on a threshold; iii) accept each of the one or more definable values which meet or exceed the threshold and reject the one or more definable values which are below the threshold; iv) employ the one or more values which meet or exceed the threshold to calculate a measurement of the feature.

In another aspect of the present disclosure there is provided a computer readable medium storing instruction for execution by a processor communicatively coupled with an ultrasound scanner, within an ultrasound imaging system, wherein when the instructions are executed by the processor, it is configured to: display, on a screen that is communicatively connected to the ultrasound scanner, an ultrasound image feed comprising ultrasound image frames comprising a feature to be measured; i) activate an AI mode in which the ultrasound scanner obtains an ultrasound signal corresponding to the feature, in whole or in part; using the AI model, identify a feature in whole or in part, and select one or more definable values of the feature on the ultrasound image, forming an AI model output; ii) calculate, using the AI model output, a validity score for the one or more definable values based on a threshold; iii) accept each of the one or more definable values which meet or exceed the threshold and reject the one or more definable values which are below the threshold; iv) employ the one or more definable values which meet or exceed the threshold to calculate a measurement of the feature; v) wherein the feature is a fetus, in whole or in part, employing the measurement to calculate a predicted gestational age of the fetus; vi) display one or more of the measurement and the predicted gestational age on the screen.

In another aspect of the present disclosure, there is provided a touchscreen device which is capable of communicating with an ultrasound scanner, the touchscreen device includes: a processor; and a memory storing instructions for execution by the processor, a interface user trigger for initiating measurements of a feature, in whole or in pat within an ultrasound image displayed on a touchscreen device, wherein when the instructions are executed by the processor, the processor is configured to: i) receive, via the touchscreen device, direction to acquire measurement of a feature by receiving inputs of an ultrasound signal of an image displayed on a screen during ultrasound scanning, said image comprising the feature within a region of interest, ii) activate an AI mode in which the ultrasound scanner obtains an ultrasound signal corresponding to the feature, in whole or in part; using the AI model, identify a feature in whole or in part, and select one or more definable values of the feature on the ultrasound image, forming an AI model output; iii) calculate, using the AI model output, a validity score for the one or more definable values based on a threshold; iv) accept each of the one or more definable values which meet or exceed the threshold and reject the one or more definable values which are below the threshold; v) employ the one or more definable values which meet or exceed the threshold to calculate a measurement of the feature; vi) wherein the feature is a fetus, in whole or in part, employing the measurement to calculate a predicted gestational age of the fetus; vii) indicate one or more of the measurement and the predicted gestational age in a manner accessible by a user (for example, viewable on touchscreen or via audible signal or other message signal).

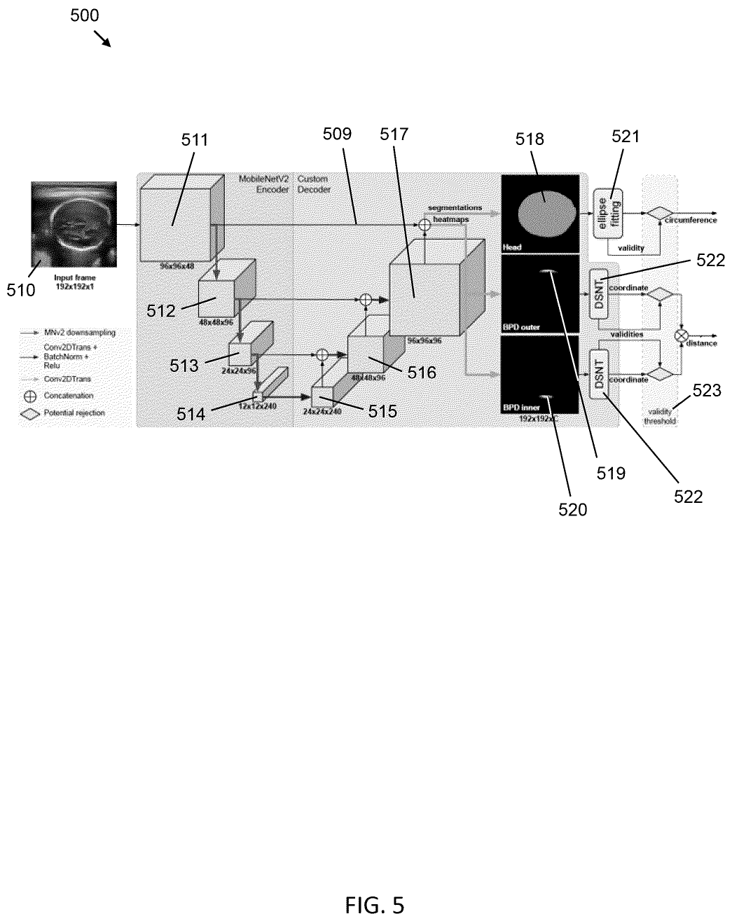

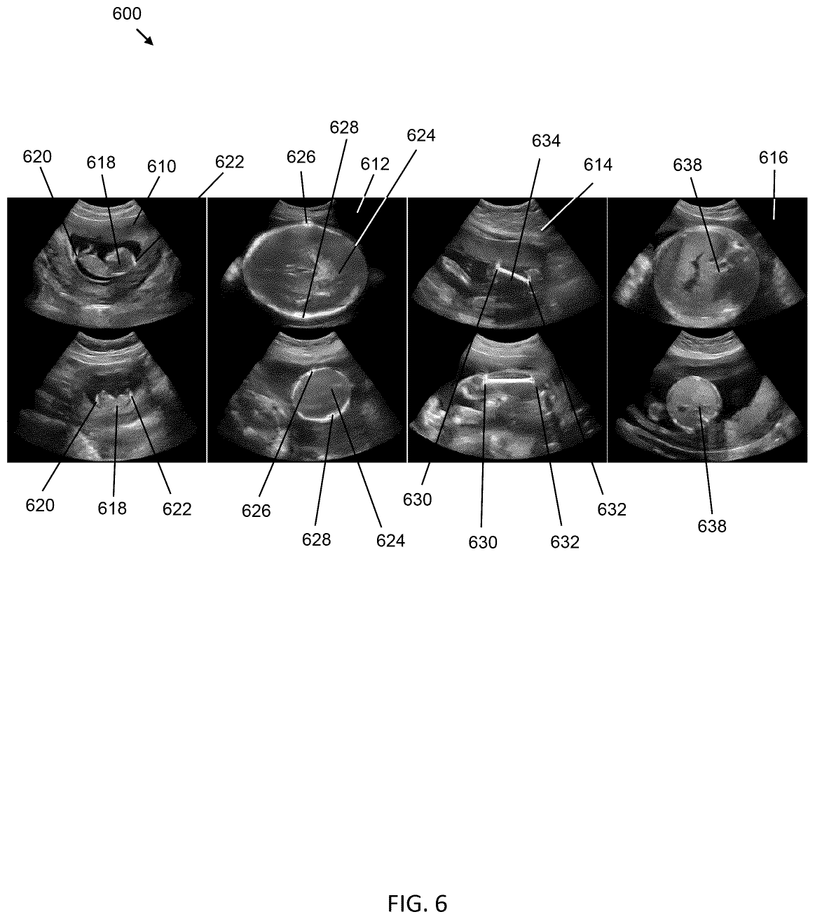

The present invention provides, in another aspect, one or more output images formed by the method of the present invention. A collection of one or more output images may comprise a visual image product, comprising measurements thereon which may be saved, collected and/or formed into a product such as, for example, a video or other media product for training and reference purposes. It is to be understood that media product as used herein comprises images, and/or videos and/or cineloops and which is rendered using a media rendering program/system, using ultrasound images generated (and optionally labelled, annotated and captioned) using the AI model and validation and measurement module/workflow of the present invention. The media product may be a video written to a physical media (such as a Compact Disc-Recordable (CD-r) or a Digital Video Disc (DVD) or made available online through cloud-based storage, through electronic communication or other transfer and data sharing means. Such media product may comprise, in addition to one or more ultrasound images, annotations and/or text overlays. In some embodiments, the media product may comprise a plurality of cineloops (see, for example, cineloop slider at 624 and play arrow 625 in FIG. 6B and FIG. 6C).

In another broad aspect of the present disclosure, there is provided a server including at least one processor and at least one memory storing instructions for execution by the at least one processor, wherein when executed, the instructions cause the at least one processor to process an ultrasound image frame against an artificial intelligence model to either classify a feature and/or segment boundaries of a feature and/or create heatmaps around a feature and/or to determine normalized co-ordinates comprising x-y co-ordinates of a centre of mass of the at least one heatmap on the ultrasound image frame (one or more defined values) and then to employ the one or more outputs of the AI model to create a validity score for one or more definable values based on a threshold, wherein the threshold may form part of a preset or may be selected or adjusted by a user; accept each of the one or more definable values which meet or exceed the threshold and reject the one or more definable values which are below the threshold; employ the one or more definable values which meet or exceed the threshold to calculate a measurement of the feature; wherein the feature is a fetus, in whole or in part, employing the measurement to calculate a predicted gestational age of the fetus; indicate one or more of the measurement and the predicted gestational age in a manner accessible by a user (for example, viewable on touchscreen or via audible signal or other message signal).

The system and method of the present invention uses a transducer (e.g., a piezoelectric or capacitive device operable to convert between acoustic and electrical energy) to scan a planar region or a volume of an anatomical feature. Electrical and/or mechanical steering allows transmission and reception along different scan lines wherein any scan pattern may be used. Ultrasound data representing a plane or volume is provided in response to the scanning. The ultrasound data is beamformed, detected, and/or scan converted. The ultrasound data may be in any format, such as polar coordinate, Cartesian coordinate, a three-dimensional grid, two-dimensional planes in Cartesian coordinate with polar coordinate spacing between planes, or other format. The ultrasound data is data which represents an anatomical feature sought to be assessed and reviewed by a sonographer. In a preferred aspect of the invention, the ultrasound image frames which are processed using the AI model of the present invention are B-mode images which are back converted, or reverse scan converted, back to their corresponding raw ultrasound data frames, or to raw ultrasound data frames in a standardized format. In this way, an operator preferably selects and freezes a post-scan converted ultrasound image, triggers the activation of the AI model (for example via or within a preset) and the ultrasound image is converted to a pre-scan converted image for processing through the AI model of the invention. Although this is a preferred mode of operation of the method of the invention, post-scan converted ultrasound images may be processed through the AI model, if trained appropriately.

Ultrasound imaging systems are becoming increasingly accessible with many current systems connecting to off-the-shelf display computing devices such as those running iOS™ or Android™ operating systems. These comprise multi-use display devices, such as, for example, tablets and smart phones. As compared to traditional ultrasound systems that have keyboards, a trackball or other physical input controls, these off-the-shelf display computing devices typically receive input via touchscreens. While the use of touchscreen input may allow for a more familiar user interface similar to what is used on consumer devices, it may be difficult to be as precise using touchscreen input versus the physical controls of traditional ultrasound systems.

Within the scope of one preferred aspect of the invention, a biometric feature may be automatically, and without additional user intervention other than activation of an AI model, identified and view-specific measurements determined, by way of a validation and measurement module. In this way, a trained, deployed AI model analyzes signals of an anatomical region of interest, in an ultrasound image frame comprising a feature as returned to the ultrasound scanner and AI model outputs comprise feature identification and/or feature segmentation and/or heat map creation and/or co-ordinate output. Depending on the structure of the feature to be measured (linear vs circumferential), an AI model of the present invention may be differently deployed and the AI model output(s) likewise different, as described further herein. Notwithstanding these differences, one or more AI model outputs, in accordance with the present invention, is processed through a validation and measurement module.

In some embodiments of the invention, ultrasound image frames are B-mode images, and it may be desired to preserve features of these images by applying optional filters thereto. For example, this can be achieved by reducing the noise levels (for example, Salt and Pepper Noise (impulse or spike noise), Poisson noise (shot noise), Gaussian or amplifier noise and Speckle Noise). This reduction may be achieved by use of one or more of the following non-limiting filter types: Gaussian filter, bilateral filter, Order statistic filter, Mean filter and Laplacian filter. It is to be understood that application of such filters is not required. As such, within the scope of the invention, additional basic parameters for the B-mode (grayscale) examination may preferably be optimized, for the acquisition of higher-quality images. These basic parameters may comprise (a) the location and number of focal zones, (b) the depth of field for ROI being imaged, (c) the two-dimensional (2D) gain setting, (d) the scan orientation, (e) the image zoom settings, and, where possible and depending on the equipment being used, (f) the presets for the specific transducer being used and the type of study being performed. These B-mode images are made viewable to a user/operator on a display screen and are adjustable as to opacity etc., A user/operator freezes a screen and pauses the acquisition of ultrasound images in order to display measurements (and gestational age, if the feature is a fetus).

### AI Model

In present invention, an artificial intelligence (AI) model is trained on a plurality of ultrasound images of anatomy/anatomical features, for the purposes described herein. As such, the present invention further provides, in another aspect, such a trained and deployable AI model.

In one aspect of the invention, a feature in an ultrasound image is segmented. There are variety methods which may be employed in AI based segmentation of ultrasound images, and the present invention is not intended to be limited to any one of these methods. Image segmentation refers to the detection of boundaries of features and structures, such as, but not limited to organs, vessels, different types of tissue in ultrasound images. In an embodiment of the present invention, a method deploys a trained AI model to perform intelligent automated recognition of segmentation tasks and intelligent automated selection and application of segmentation algorithms. This allows the AI model to be applied to intelligently perform various different segmentation tasks, including segmentation of a fetus, in different views. The AI model can intelligently select one or a combination of segmentation algorithms from a plurality of segmentation algorithms to perform appropriate segmentation for various features and anatomical object. For example, the algorithms may be a threshold-based segmentation algorithm, an edge-based segmentation algorithm, a region-based segmentation algorithm, a clustering-based segmentation algorithm, or the like, or a combination thereof.

In some embodiments of the invention, segmentation algorithms may be stored in a segmentation algorithm database which may comprise a plurality of deep learning-based ultrasound image segmentation methods, each of which including a respective trained deep neural network architecture for performing ultrasound image segmentation. For example, the segmentation algorithms can include the deep learning based segmentation algorithms described below, including segmentation using a deep neural network (DNN) that integrates shape priors through joint training, non-rigid shape segmentation method using deep reinforcement learning, segmentation using deep learning based partial inference modeling under domain shift, segmentation using a deep-image-to-image network and multi-scale probability maps, and active shape model based segmentation using a recurrent neural network (RNN). The segmentation algorithm database may include other deep learning-based segmentation algorithms as well, such as marginal space deep learning (MSDL) and marginal space deep regression (MSDR) segmentation methods. It is also possible that a segmentation algorithm database may also store various other non-deep learning-based segmentation algorithms, including but not limited to machine-learning based segmentation methods (e.g., marginal space learning (MSL) based segmentation), graph cuts segmentation methods, region-growing based segmentation methods, and atlas-based segmentation methods.

A segmentation algorithm database may store multiple versions of each segmentation algorithm corresponding to different target anatomical features and structures. For deep learning-based segmentation algorithms, each version corresponding to a specific target anatomical structure may include a respective trained deep network architecture with parameters (weights) learned for segmentation of that target anatomical structure. For a particular anatomical structure, a segmentation algorithm database can also store multiple versions corresponding to different imaging domains and/or image quality levels. For example, different deep learning architectures can be trained and stored using images with different signal-to-noise ratios. Accordingly, when a master segmentation artificial agent selects one or more segmentation algorithms from the those stored in a segmentation algorithm database, the master segmentation artificial agent may select not only the type of segmentation algorithm to apply, but the specific versions of segmentation algorithms that are best for performing the current segmentation task.

In some embodiments, the ultrasound frames of a new ultrasound image, imaged in ultrasound imaging data may be processed against an AI model on a per pixel basis, and thus the segmentation of boundaries of features, in whole or part, on the new ultrasound image, thereby creating one segmented boundary feature or two or more segmented boundary features, imaged in new ultrasound imaging data, may be generated on a per pixel basis. When deployed, an output of the AI model for a first pixel of the new ultrasound imaging data may be used to corroborate the output of the AI model for a second pixel of the new ultrasound imaging data adjacent or within the proximity to the first pixel.

Alternatively, the ultrasound frames of new ultrasound images, imaged in ultrasound imaging data may be processed against an AI model on a line/sample basis, and the and thus the segmentation of boundaries of the feature or features, in whole or part, on the new ultrasound image, thereby creating at least one or two or more segmented boundary features, imaged in new ultrasound imaging data, may be generated on a line/sample basis.

Image segmentation algorithms may automatically identify structures, such as a fetus, in whole or part, in ultrasound images. An example of such a traditional approach is to use an active contours model (also called a “snakes” algorithm) to delineate the outline of the oblong shape visually identified by the ultrasound operator. However, using the active contours model algorithm requires complex mathematical calculations involving the solving of partial differential equations. This is a computationally intensive process. While performing this type of process on a traditional cart-based ultrasound system with high computational and power capacity may not be a problem, executing this type of algorithms on a touchscreen device that connects to an ultra-portable ultrasound scanner may be more difficult. This is because the touchscreen device may be limited in processing ability and battery power; such that executing these types of traditional algorithms on a touchscreen device may result in lower responsiveness in the user interface of the ultrasound application executing on the touchscreen device.

Instead of using a traditional active contours model algorithm, the present embodiments may use a contour identification process that uses morphological operators. For example, to perform morphological processing on an image, an image may first be thresholded to generate a binary image. Then, a structuring element (for example, a small binary configuration of pixels that could be in the shape of a cross or a square) may be positioned at all possible locations of the binary image, to generate a secondary binary image. As each structuring element is positioned over the first binary image, how the structuring element relates to the underlying pixels of the first binary image impacts whether a pixel location is set to ‘1’ or ‘0’ in the secondary binary image.

For example, two common morphological operations are “erosion” and “dilation”. In erosion, as the structuring element is positioned over the possible locations of the first binary image, it is required that all the ‘1’ pixels in the first binary image “fit” into the structuring element for the corresponding pixel locations on the second image to be set to ‘1’. On the edges of any structures appearing on the first binary image, it will generally not be possible to meet this requirement because there will be a combination of ‘1’s and ‘0’s in the structuring element. This will result in some of those pixels that were set to ‘1’ in the first binary image being set to ‘0’ in the second binary image. In this manner, this operation results in a layer of pixels being “eroded” away in the second binary image.

In dilation, as the structuring element is positioned over the possible locations of the first binary image, it is only required that the structuring element “hit” any of the ‘1’ pixels (e.g., that at least one of the pixels in the structuring element is a ‘1’) for the corresponding pixel locations on the second image be set to ‘1’. On the edges of any structure appearing in the first binary image, there will again generally be a combination of ‘1’s and ‘0’s in the structuring element. However, unlike erosion, the requirement for the structuring element to “hit” a ‘1’ pixel will be met. This will result in some of those pixels that were set to ‘0’ in the first binary image be changed to a ‘1’ in the second binary image. In this manner, this operation results in a layer of pixels being added, and thus the structure in the first binary image is “dilated” in the second binary image.

These types of morphological operators can be used in a contour identification process. For example, a morphological snakes algorithm is similar to the traditional active contours model or “snakes” algorithm, except that morphological operators (e.g., dilation or erosion) are used to grow or shrink the contour. Since morphological operators operate generally on binary images, operations can be performed over a binary array instead of over a floating-point array (as would be the case if a traditional active contours model or “snakes” process is used). Thus, using a contour identification process that uses morphological operators may be considered less computationally intensive, and such processes may be particularly suitable for execution with ultrasound scanners that connect to mobile touchscreen devices that generally have lower computational capabilities and operate on battery power. This mode of contour identification is described in U.S. Pat. No. 11,593,937, the entire contents of which are incorporated herein by reference.

In one embodiment of the invention, the AI model comprises a U-Net model with MobileNetV2 backbone (MNV2 U-Net). This architecture allows the model to automatically learn hierarchical representations from the input images, effectively capturing fine details and spatial relationships within the anatomy of interest. A U-Net is a type of convolutional neural network (CNN) architecture specifically designed for image segmentation tasks wherein the name “U-Net” is derived from the U-shaped structure of the network, which consists of an encoding (contracting) path and a decoding (expanding) path. The U-Net architecture can be divided into two main parts:
(a) Contracting path (encoder): The encoder is composed of a series of convolutional layers followed by max-pooling layers. These layers help the network learn to capture the local features and context of the input image while progressively reducing the spatial dimensions. Moving deeper into the encoder, the feature maps become smaller but have more channels, encoding more complex features.
(b) Expanding path (decoder): The decoder part of the U-Net takes the output from the encoder and reconstructs the input image's spatial dimensions through a series of up-convolution (also known as transposed convolution) layers followed by concatenation with the corresponding feature maps from the encoder. This step-by-step expansion and concatenation of the feature maps enable the network to precisely localize and segment objects in the image.

A key feature of the U-Net architecture is the skip connections between the encoder and decoder parts. These connections enable the transfer of high-resolution spatial information from the encoding path to the decoding path, allowing for better localization and segmentation of objects in the image. U-Nets have been widely adopted and adapted for various image segmentation tasks and has an ability to efficiently learn from a small number of labeled images while providing accurate and precise segmentation results.

MobileNet is a family of lightweight and efficient CNNs designed specifically for mobile and embedded devices, where computational resources and power consumption are limited. The key innovation in MobileNet architecture is the use of depth wise separable convolutions, which significantly reduces the number of parameters and computations compared to traditional CNNs. Depth wise separable convolutions divide the standard convolution operation into two separate operations: 1) depth wise convolutions, which apply filters to each input channel independently, and 2) pointwise convolutions, which combine the outputs of depth wise convolutions using 1×1 filters. This design choice allows MobileNet to maintain a high level of accuracy while being computationally efficient and lightweight.

A U-Net model with a MobileNetV2 backbone is a variant of the U-Net that replaces the original encoder with the MobileNetV2 architecture. MobileNetV2 is a CNN that is designed to be lightweight and efficient, making it suitable for mobile and embedded vision applications. Its main characteristic is the use of inverted residuals and linear bottlenecks that help in decreasing the number of parameters and computational cost, without affecting the performance of the model.

The following are different parts of the U-Net model with a MobileNetV2 backbone.
Encoder (MobileNetV2): The purpose of the encoder is to extract features from the input image. In this model, the encoder is replaced with the MobileNetV2 architecture. The MobileNetV2 has 17 layers with residual connections. These residual connections help to train deeper networks by allowing gradients to flow through the network from the output end to the input end during backpropagation.
Bottleneck: After the encoding stage, the model consists of a bottleneck layer. This layer is often a convolutional layer that further processes the feature maps.
Decoder: The decoder takes the feature representation from the bottleneck layer and upsamples it to produce an output that is the same size as the input. In the U-Net architecture, this is done by a series of transposed convolutions. Importantly, the decoder also takes as input the feature maps from the encoder at the same level (these are usually concatenated or added together), which gives the decoder access to the spatial information lost during down sampling.
Skip connections: Skip connections are a key part of the U-Net architecture. They involve passing the output of a layer from the encoder directly to the corresponding layer in the decoder, which helps to retain the high-resolution features and spatial context, enabling precise localization.

When deployed, U-Net with MobileNetV2 backbone provides a good balance between performance and computational efficiency, as MobileNetV2 is designed to be small and fast, while U-Net's architecture is well suited to image segmentation tasks.

The trained AI model of the present invention produces outputs including one or more segmentation outputs and one or more landmark outputs. Depending on the feature to be identified, and measured/validated, different outputs of the AI model may be employed by the validation/measurement module of the present invention. For example, in an obstetric examination, this will, to some extent, be dependent on a user/operator selection of either early OB ultrasound (such as, for example, up to 12-13 weeks gestation) or later OB ultrasound (such as, for example, after 13 weeks gestation). It is to be understood that these week points for early and late may be adjusted (i.e., what is early versus late), depending on patient and ultrasound user/operator. In ultrasound images of a fetus in earlier points in gestation, commonly AI model outputs may be a segmentation of the fetus and the measurement obtained by the method of the invention is the validated linear CRL, from which gestational age may be calculated. In ultrasound images of a fetus in later points in gestation, commonly the AI model output may be one or more of a segmentation of HC and/or AC, the measurements obtained by the method of the invention is one or more of a validated circumference of HC and/or AC and the validated linear distances BPD and/or FL, from which gestational age may be calculated.

### AI Model Outputs

When an anatomical feature or ROI within an ultrasound image comprises a fetus, or part thereof, three of the five measurements (CRL, FL, and BPD) and optionally gestational age, are determined within the scope of the invention by identifying and validating a landmark target. When a feature to be measured comprises a landmark target, there are preferably at least two AI model outputs: 1) a heatmap, and 2) a coordinate output, as described below such outputs then being processed through the validation/measurement module. When an anatomical feature or ROI within an ultrasound image comprises a fetus, or part thereof, two of the five common measurements (HC and AC) and optionally gestational age, are preferably determined, within the scope of the invention, by identifying, validating and measuring a segmentation mask.

### AI Model Landmark Outputs

When a feature to be measured comprises a landmark target, there are preferably at least two AI model outputs: 1) a heatmap, and 2) a coordinate output. The heatmap represents a likelihood mapping with the same size as the input image, where each pixel's value represents the likelihood that the landmark is located at that pixel. The ground truth labels for these heatmap are preferably generated by creating a 2D Gaussian “blob” centered around a ground truth landmark location. The heatmap outputs are treated the same as the segmentation outputs (described above) and use the same sigmoid output activation.

When an anatomical feature or ROI within an ultrasound image comprises a fetus, or part thereof, three of the five common measurements (CRL, FL, and BPD) are produced using coordinates directly regressed from images processed using the AI model of the invention. In one embodiment, a Differentiable Spatial to Numerical Transform (DSNT) layer may be added to a fully convolutional network for the task of coordinate regression. The DSNT layer transforms spatial heatmaps into numerical coordinates using a fully differentiable method, allowing it to be included in end-to-end training schema. To create a ground truth (GT) label for a landmark output, in one embodiment, a 2D Gaussian heatmap is generated, centered around a labelled coordinate location. It has been found to be preferable to skew such heatmaps such that they are wider along the short axis (perpendicular to the measurement) which leads to better results as it penalizes long-axis errors more aggressively. Additionally, the size of the generated heatmaps are scaled by the length of the measurement such that longer measurements result in larger heatmap. Samples of GT heatmaps are shown in FIG. 5 which illustrates exemplary fetal images with GT labels. From left to right: 1) Fetus segmentation with CRL Head/Rump landmarks; 2) Head segmentation with BPD inner/outer landmarks; 3) Femur segmentation with FL distal/proximal landmarks and 4) Abdomen segmentation (no associated landmarks).

Within the scope of the invention a DSNT layer essentially outputs the x-y coordinates of the center-of-mass of the input heatmap, normalized between 0-1, with 0-0 representing the top left pixel of the image, and 1-1 representing the bottom right. In this way, there may be implemented a validity metric, measurable by way of the validity module of the invention.

Within the scope of the present invention, the landmark heatmaps (produced as described above) are passed to DSNT layers, within the AI model, which output coordinate values. In one embodiment, the method of the invention takes advantage the differentiable nature of the DSNT layer by including Euclidean distance loss for all coordinate outputs in a loss calculation. Dice loss may also be included for the segmentation outputs, mean squared error for the heatmaps, and categorical cross-entropy loss for the view classification using, for example, the following Equation A.1:

$$\text{?} = \text{?} + \text{?} + \text{?} \qquad ( A \text{.1} )$$

$$\text{?} = 1 - \frac{2 \text{?}}{\text{?} + \text{?} + ϵ}$$

$$\text{?} = \sqrt{{( \text{?} - \text{?} )}^{2} + {( \text{?} - \text{?} )}^{2}}$$

$$\text{?} = \frac{1}{N} \text{?} {( \text{?} - \text{?} )}^{2}$$

$$\text{?} \text{indicates text missing or illegible when filed}$$

wherein where Ls is the segmentation loss (Dice loss), Lc is the coordinate loss (Distance loss), Lh is the heatmap loss (MSE), y is the ground truth value, \hat{ }y is the prediction, ϵ is a small smoothing factor, N is the number of pixels in an image. To weigh the different loss types, we use αc=10 for the coordinate, and αh=500 for the heatmaps as the MSE loss is very small compared to the other two types.

Including the heatmap loss term encourages the AI model of the invention to converge more quickly, while the coordinate loss term improves the final accuracy.

### AI Model Segmentation Mask Outputs

When a feature to be measured comprises a circumferential target, such as for example a of a parietal structure, the AI model output is a segmentations masks, for example, in an ultrasound image comprising a fetus, of the abdomen and/or head, which are used to produce the AC and HC measurements respectively. Segmentation “mask” refers to a region in the ultrasound image highlighting the ROI, for example a fetus in whole or part. The process of segmentation has two parts:
1. Localization: Identification of the fetus and fetal anatomies in an ultrasound image; and
2. Pixel-wise classification: Identification of pixels belonging to the fetus and labelling them as 1s and the rest of the pixels as 0s.
   The segmentation mask is overlayed onto the ultrasound images during the imaging process, thereby locating the distinct fetal anatomies.

### Validation and Measurement Module/Workflow

The present invention additionally comprises a validation and measurement module which receives one or more AI model outputs, validates one or more of those outputs against a threshold, applies onto the ultrasound image the appropriate number of caliper sets only if the threshold(s) is met or exceeded; and ii) acquires measurements from the applied caliper set(s). Additionally, the validation and measurement module may automatically calculate gestational age using one or more of the acquired measurements. Additionally, the validation and measurement module may automatically prompt a user to adjust an ultrasound scanner to either i) acquire additionally views of the ROI in additional ultrasound image frames so as to ensure the capture of a view most suitable for acquiring measurement(s); and/or ii) scroll cineloop for additionally views of the ROI in additional ultrasound image frames so as to ensure the capture of a view most suitable for acquiring measurement(s).

AI outputs undergo post-processing, in the validity/measurement module, to produce clinically relevant measurements. In this module, a “validity” value is calculated for various AI output types: landmark and segmentation. The validity of a given landmark may be calculated by measuring a local heatmap intensity around a regressed coordinate. A high-validity landmark will have a bright, focused heatmap, with the predicted coordinate located amid the most intense pixels, while a low validity landmark will have a dispersed heatmap with low intensity surrounding the coordinate. This also serves to filter out a common DSNT error mode; wherein the model splits its prediction between two locations in the image (e.g. the two ends of a femur bone); both get bright heatmaps, however the coordinate calculated by the DSNT layer lies close the middle of the two. Because the heatmap at that location is basically zero, cases such as these are appropriately filtered out by the preferred method of the invention. The exact calculation for validity can be formulated using, for example, Equation A.0:

$$\text{?} = \frac{\text{?}}{\text{?}} \text{?} ( σ \text{?} \sqrt{\text{?} + \text{?}} )$$

$$\text{?} \text{indicates text missing or illegible when filed}$$

wherein W and H are the width and height of the image, Mij is the heatmap value at the (i, j) coordinate, G1D(σ, . . . ) is the 1D Gaussian kernel with width σ, (xo, yo) the coordinate output by the DSNT layer, and Aσ a normalization constant equal to the area under a 1D Gaussian curve with width σ.

Any coordinate with validity below a predetermined threshold is rejected and a measurement is not taken (ex: no caliper set placed or only caliper placed). Any coordinate with validity at or above a predetermined threshold is accepted. The following table shows which network outputs are associated with which target measurements along with their respective validity thresholds. If both landmarks that form a distance-measurement pair are valid (ex: distance measurement pairs found at each end of a femur, at each of crown and rump or at each opposing side of a parietal structure), the Euclidean distance between them is calculated and displayed that to a user, such as, for example, on a display screen. Measurements may be converted from the pixel domain to the physical domain using the millimeter pixel spacing, which is saved for example, in the ultrasound scanner metadata. Pre-scan to post-scan conversion of the mages may then be performed, as it is preferred (although not required) that the AI model of present invention is trained on and processes pre-scan converted images, while the final output of the method and system of the invention may display post-scan converted images to the user.

By way of example, the method steps, post-processing, for one or more landmark outputs of an AI model (which always come in pairs) for measuring the physical length of anatomical structures in the input images (ex: three landmark-based measurements comprise CRL, BPD, and FL), the post-processing steps are as follows:
1. Scale the normalized coordinates (0-1) back up to the original image size (in pixels);
2. Calculate the landmark validity as the intensity of the heatmap pixels surrounding the predicted coordinate;
3. If both landmarks are valid, calculate the Euclidean distance between the coordinates (in pixels); and
4. Convert the pixel-distance to physical distance (in mm) using pixel spacing information from the probe's metadata.

The validity of a given circumferential or parietal feature (ex: AC and HC) may be calculated by matching or overlay of a segmentation mask (ex; of AC and/or HC) to an ellipse. An ellipse is fit onto the largest contour in a segmentation mask, the circumference of which forms a measurement value. A respective validity value is calculated as the overlap between the fit ellipse area and the predicted contour area. A high-validity segmentation will appear nearly elliptical in shape, while a low-validity prediction may contain holes or concavities. Circumference measurements for the circumferential or parietal feature with validity below a predetermined threshold are rejected and are not displayed to the user. By way of example, the method steps, post-processing, for two segmentation-based measurements (AC and HC), are as follows:
1. Find the contour of the largest connected component in a predicted segmentation mask;
2. Fit an ellipse around the contour of the connected component;
3. Calculate a validity score as the nsgeormalize overlap between the ellipse and the original predicted mask.
4. Calculate the circumference (in mm) of the ellipse if the validity is at or above a threshold.

There are several metrics for evaluating the performance of the AI model of the present invention, including, without limitation, the Jaccard index: This similarity function is used to measure how closely the contours of segmentation out-puts predicted by the AI model align with those of the ground-truth labels. This metric is used to evaluate the performance of all four (4) segmentation outputs (Fetus, Abdomen, Femur, and Head). The Jaccard Index, also known as the Jaccard Similarity Coefficient, or Intersection Over Union (IoU), is computed using the formula:

$$J ( A , B ) = \frac{\vert A ⋂ B \vert}{\vert A ⋃ B \vert}$$

Wherein A is the segmentation mask predicted by the model and B is the ground-truth mask.

### Threshold Selection

Validity thresholds (also referred to as threshold values), for use within the validity module, may be pre-selected within a preset or workflow or may be selected and adjusted by a user/operator (or a user may adjust an already pre-selected validity threshold, as desired for a particular clinical or diagnostic goal). Adjustment of a validity threshold effects the precision and recall of predictions, within the method of the invention. For example, increasing the minimum validity threshold for a certain measurement will allow for more positive true predictions (increasing recall) but will also result in more false positive predictions (decreasing precision). Thus, on many occasions, when selecting a threshold value, high precision may be preferred over high recall. This ensures that the number of false positive predictions are limited, as those could confuse the end-user and potentially lead to incorrect clinical decision making. A secondary consequence of this is that fewer measurements will be accepted as valid, reducing the number of predictions displayed to the user. This is a representation of a network not being confident in its predictions, which could lead to inaccurate measurements, information which is valuable to a user.

Confusion matrices are shown below in Table 1, where a measurement is considered predicted if its validity value is above that measurement's validity threshold. Maintaining a high precision value ensures that the system does not produce too many false positive predictions, while a high recall value ensures that system does not miss too many false negatives. For each measurement, there is a trade-off between these two metrics, controlled by the selection of the validity threshold. As the threshold is increased, more low validity samples are rejected, resulting in fewer positive predictions, i.e. higher precision and lower recall. While tuning the threshold values, maximizing precision is preferred over recall, as false positive cases can be confusing for the end-user, and can even result in incorrect decision making.

In order to ensure the AI model accurately identify cases with/without the anatomies of interest, within the design of the method of the invention, precision and recall were also monitored for each output. These two metrics were used to evaluate the performance of all segmentations and measurements (Fetus, Abdomen, Femur, Head, AC, HC, CRL, BPD, and FL). Precision measures the proportion of positively predicted samples that contain the target anatomy, whereas recall measures the proportion of samples containing the target anatomy that the model produced predictions for. They are calculated as follows:

$$Precision = \frac{tp}{tp + fp}$$

$$Recall = \frac{tp}{tp + fn}$$

### Wherein

tp: Number of True Positives. Samples with model prediction and ground-truth label
fp: Number of False Positives. Samples with model prediction, but no ground-truth label
fn: Number of False Negatives. Samples with a ground-truth label, but no model prediction

Ideally, metrics should be as close to 1 as possible, however, as described above, there is a balance or trade-off between the two, which may be driven by specific scanning, particular anatomy, a user preference, clinical or diagnostic goals.

TABLE 1

| | | CRL | | | FL | | | BPD |
|---|---|---|---|---|---|---|---|---|
| Ground | TRUE | 261 | 170 | TRUE | 534 | 67 | TRUE | 497 |
| Truth | FALSE | 76 | 3805 | FALSE | 2 | 4202 | FALSE | 22 |
| | | TRUE | FALSE | | TRUE | FALSE | | TRUE |

| | | |
|---|---|---|
| Predicted | Predicted | Predicted |

| | BPD | | HC | | | AC | |
|---|---|---|---|---|---|---|---|
| Ground | 14 | TRUE | 495 | 26 | TRUE | 417 | 148 |
| Truth | 4053 | FALSE | 9 | 4065 | FALSE | 7 | 3909 |
| | FALSE | | TRUE | FALSE | | TRUE | FALSE |

| | | |
|---|---|---|
| Predicted | Predicted | Predicted |

wherein what is shown is the number of cases with/without GT measurements vs. with/without predicted measurements.

By way of example only, Table 2 shows outputs associated with target measurements along with exemplary, respective validity thresholds:

TABLE 2

| | | | Minimum |
|---|---|---|---|
| Name | Type | Associated Measurement | validity |
| Fetus | segmentation | n/a | n/a |
| Abdomen | segmentation | Abdominal Circumference (AC) | 0.02 |
| Femur | segmentation | n/a | n/a |
| Head | segmentation | Head Circumference (HC) | 0.02 |
| CRL_coords | landmarks | Crown Rump Length (CRL) | 0.005 |
| BPD_coords | landmarks | Biparietal Diameter (BPD) | 0.01 |
| FL_coords | landmarks | Femur Length (FL) | 0.1 |

In one aspect of the invention, one or more measurements and/or gestational age are displayed visually on an interface, such as an interface on a multi-purpose electronic device. This visual display may provide, for example the actual measurement number (ex: CRL, FL, BPD, AC and HC and “age”) encircled or in a prominent, easy to view area of the interface. In another aspect of the invention, the one or more measurement numbers may be conveyed to a user audibly. In using presets or other AI enhancement modules, such an interface may also convey to a user, visually or audibly, the AI model identified/predicted view being scanned and subsequently the measurements thereafter acquired.

For simplicity and clarity of illustration, where considered appropriate, reference numerals may be repeated among the figures to indicate corresponding or analogous elements or steps. In addition, numerous specific details are set forth in order to provide a thorough understanding of the exemplary embodiments described herein. However, it will be understood by those of ordinary skill in the art that the embodiments described herein may be practiced without these specific details. In other instances, certain steps, signals, protocols, software, hardware, networking infrastructure, circuits, structures, techniques, well-known methods, procedures and components have not been described or shown in detail in order not to obscure the embodiments generally described herein.

Furthermore, this description is not to be considered as limiting the scope of the embodiments described herein in any way. It should be understood that the detailed description, while indicating specific embodiments, are given by way of illustration only, since various changes and modifications within the scope of the disclosure will become apparent to those skilled in the art from this detailed description. Accordingly, the specification and drawings are to be regarded in an illustrative, rather than a restrictive, sense.

The system of the present invention uses a transducer (e.g., a piezoelectric or capacitive device operable to convert between acoustic and electrical energy) to scan a planar region or a volume of an anatomical feature. Electrical and/or mechanical steering allows transmission and reception along different scan lines wherein any scan pattern may be used. Ultrasound data representing a plane or volume is provided in response to the scanning. The ultrasound data is beamformed, detected, and/or scan converted. The ultrasound data may be in any format, such as polar coordinate, Cartesian coordinate, a three-dimensional grid, two-dimensional planes in Cartesian coordinate with polar coordinate spacing between planes, or other format. The ultrasound data is data which represents an anatomical feature sought to be assessed and reviewed by a sonographer.

A user input device may comprise one or more of a touchscreen, a keyboard, a mouse, a trackpad, a motion sensing camera, or other device configured to enable a user to interact with and manipulate data within image processing system. In one example, user input device may enable a user to make a selection of an ultrasound image to use in training an AI model, or for further processing using a trained AI model. A display device may include one or more display devices utilizing virtually any type of technology. In some embodiments, display device may be part of a multi-purpose display device or may comprise a computer monitor, and in both cases, may display ultrasound images. A display device may be combined with processor, non-transitory memory, and/or user input device in a shared electronic device, or there may be peripheral display devices which may comprise a monitor, touchscreen, projector, or other display device known in the art, which may enable a user to view ultrasound images produced by an ultrasound imaging system, and/or interact with various data stored in non-transitory memory.

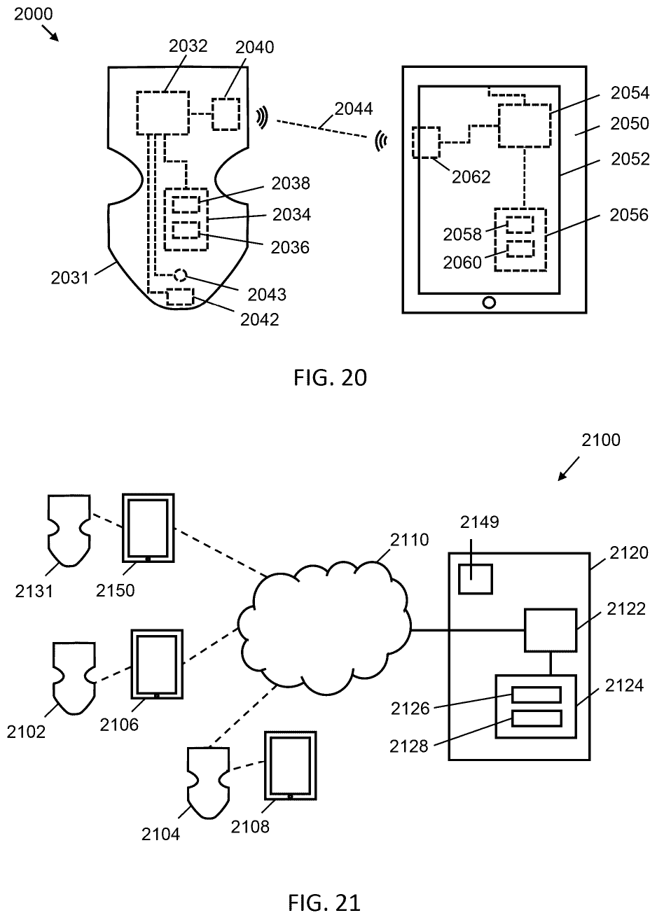

In various embodiments, a multi-purpose electronic devices/display devices may be, for example, a laptop computer, a tablet computer, a desktop computer, a smart phone, a smart watch, spectacles with a built-in display, a television, a bespoke display or any other display device that is capable of being communicably connected to an ultrasound probe. Multi-purpose electronic devices/display devices may host a screen (such as shown in FIGS. 20 and 21), and may include a processor, which may be connected to a non-transitory computer readable memory storing computer readable instructions, which, when executed by the processor, cause the display device to provide one or more of the functions of the system (such system comprising at least one multi-purpose electronic device and at least probe). Such functions may be, for example, the receiving of ultrasound data that may or may not be pre-processed; scan conversion of received ultrasound data into an ultrasound image; processing of ultrasound data in image data frames; the display of a user interface; the control of a probe and the display of an ultrasound image on the screen. Such a screen may comprise a touch-sensitive display (e.g., touchscreen) that can detect a presence of a touch from the operator on screen and can also identify a location of the touch in screen. The touch may be applied by, for example, at least one of an individual's hand, glove, stylus, or the like. As such, the touch-sensitive display may be used to receive an input, for example, indicating the presence or absence of text or annotations on an image. The screen and/or any other user interface may also communicate audibly. Multi-purpose electronic devices/display devices may be configured to present information to the operator during or after the imaging or data acquiring session. The information presented may include ultrasound images (e.g., one or more 2D frames), graphical elements, measurement graphics of the displayed images, user-selectable elements, user settings, and other information (e.g., administrative information, personal information of the patient, and the like).

Also stored in the computer readable memory within the multi-purpose electronic devices/display devices may be computer readable data which may be used by processors within multi-purpose electronic devices/display devices, in conjunction with the computer readable instructions within multi-purpose electronic devices/display devices (ex: 2050 in FIG. 20), to provide the functions of the system. Such computer readable data may include, for example, settings for ultrasound probe, such as presets for acquiring ultrasound data and settings for a user interface displayed on screens. Settings may also include any other data that is specific to the way that the ultrasound probe operates or that multi-purpose electronic devices/display devices operate.

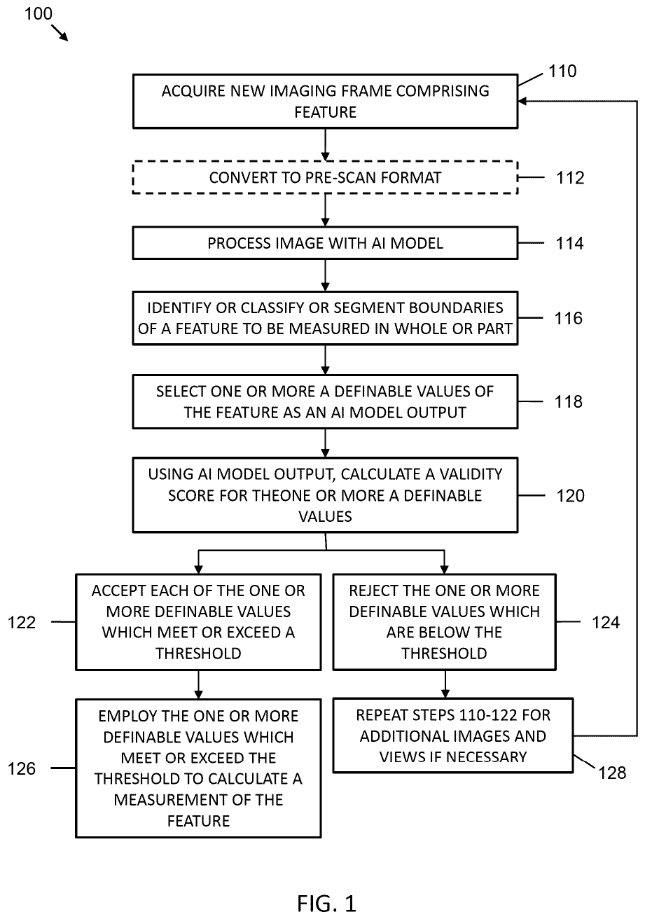

Referring to FIG. 1, there is shown a flowchart diagram of a method, generally indicated at 100, of new image frame acquisition of a region of interest comprising a feature; deploying an AI model to execute on a computing device communicably connected to the ultrasound scanner, wherein the AI model is trained so that when the AI model is deployed, the computing device identifies a feature to be measured, in whole or part and selects a definable value of the feature, which is an AI model output. At 110, the ultrasound imaging system (e.g., as referred to in FIGS. 20 and 21), may acquire ultrasound imaging data in the form of an imaging frame. For example, a user may operate an ultrasound scanner (hereinafter “scanner”, “probe”, or “transducer” for brevity) to capture images of a patient. The ultrasound frames (B-mode) may be acquired by acquiring a series of a images (with a frame each containing a sequence of transmitted and received ultrasound signals) of different views of the abdominal region. The performance of the AI model within the scope of the invention may be optimized by the removal of common ultrasound artifacts like an acoustic shadow, lateral shadow, anisotropic effect, reverberation, and refraction. As these artifacts are common in ultrasound, users may optionally adopt one or more of the following techniques to reduce these artifacts during use, such as, for example: scanning from different angles and planes and ensuring the anatomy of interest is centered and in focus.

Further, at step 112, a new ultrasound imaging frame may optionally be pre-processed and/or augmented. In some embodiments, an optional pre-processing act may be performed on the new ultrasound image frame to facilitate improved performance and/or accuracy when training the machine learning (ML) algorithm and when deploying the machine learning (ML) algorithm through the AI model. For example, it may be possible to pre-process the ultrasound imaging frame through a high contrast filter to reduce the granularity of greyscale on the ultrasound image. Additionally, or alternatively, it may be possible to reduce scale of the ultrasound image frame prior to providing the ultrasound image frame for processing through the AI model at step 114. Reducing the scale of ultrasound image frame as a preprocessing step may reduce the amount of image data to be processed, and thus may reduce the corresponding computing resources required. Various additional or alternative pre-processing acts may be performed. For example, these acts may include data normalization to ensure that the various ultrasound imaging frame has the dimensions and parameters which are optimal for processing through the AI model. All such processing may be prior to display of an ultrasound image for viewing to a user. At step 114, the ultrasound image may be converted to pre-scan format for the purpose of processing through the AI model.

At step 114, the new ultrasound imaging frame/image data is processed with an AI model so that at step 116, a feature to be measured is identified and/or classified and/or segmented by boundaries and/or by segmentation mask, in whole or in part. As described herein, such identification, classification, segmentation etc., may be achieved by a variety of methods, including, but not limited to, segmentation of boundaries/edge detection, contouring and classification. This invention is not intended to be limited to any one mode of AI-model-generated feature identification. The product of the AI model is one or more definable values of the feature, selected by the AI model at step 118. The output is automatically conveyed to a validity and measurement module at step 120 which determines a validity score for the one or more definable values. At step 122, each of the one or more definable values which meet or exceed a threshold is accepted and at step 124, each of the one or more definable values which are below the threshold are rejected. Each of the one or more definable values which meet or exceed a threshold is, at step 126, employed to calculate a measurement of the feature. Each of the one or more definable values which are below the threshold is, at step 128, rejected and steps 100-120 may be repeated, as illustrated by the arrow and line back to step 110. Such redirection enables other images to be selected of the feature, and may allow better images to be processed, in order to acquire the desired measurement. Such new images may not require rescanning but could be selected from a cineloop.

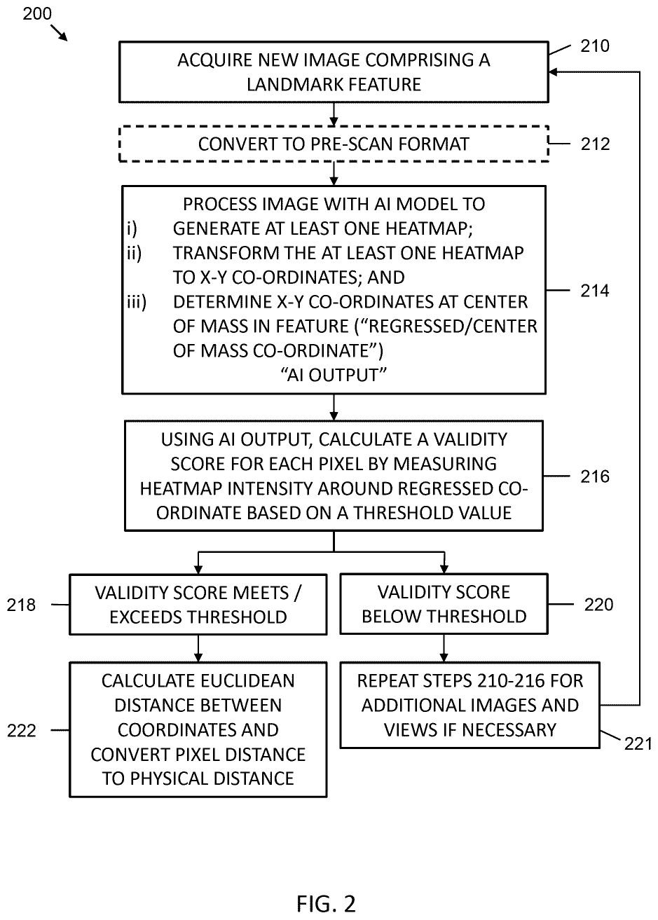

A flowchart diagram of a method in accordance with one embodiment of the invention, generally indicated at 200, is shown in FIG. 2. At step 210, a new ultrasound image comprising a landmark feature is acquired and at step 212, the new ultrasound imaging frame may optionally be pre-processed and/or augmented prior to display for viewing to a user. Prior to deployment through the AI model of the invention, such image may be converted to a pre-scan format. The AI model, at step 214 engages in processing the pre-scan converted image and generates at least one heatmap, transforms the at least one heatmap to x-y co-ordinates and determines the x-y co-ordinates at a center of mass in the feature (“regressed center of mass co-ordinate”), which is at least one of AI module outputs. The AI model output, at step 216 is employed by a validity module to calculate a validity score of each pixel by measuring heatmap intensity around a regressed co-ordinate based on a threshold. At step 218, when a validity score meets or exceeds a threshold, it is accepted, and subsequently at step 222 a Euclidean distance between co-ordinates if calculated and thereafter pixel distance converted to physical distance. Conversely, when a validity score is below a threshold, shown at step 220, the validity module directs the repeat of steps 210-216 as illustrated by the arrow and line back to step 210. Such redirection enables other images to be selected of the feature, and may allow better images to be processed, in order to acquire the desired measurement. Such new images may not require rescanning but could be selected from a cineloop and processed through steps 210-216.

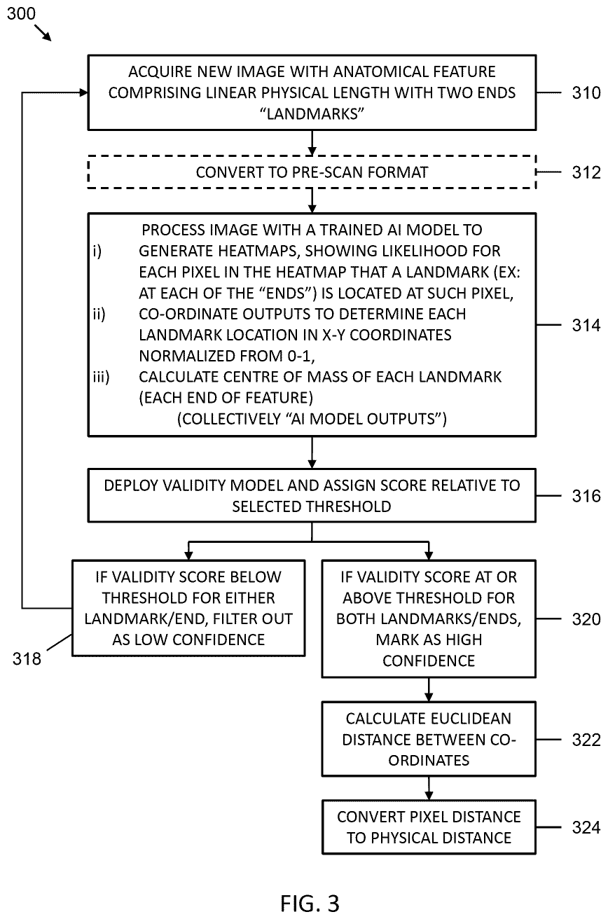

A flowchart diagram of a method in accordance with one embodiment of the invention, generally indicated at 300, is shown in FIG. 3. At step 310, a new ultrasound image is acquired comprising an anatomical feature comprising a linear physical length desired to be measured with two “end” or landmarks. It is to be understood such new ultrasound image may be processed prior to display of an ultrasound image for viewing to a user, and, at step 312, the ultrasound image may be converted to pre-scan format for the purpose of processing through an AI model of the invention. The AI model, at step 314 engages in processing the image and generates at least one heatmap showing likelihood for each pixel in the heatmap that a landmark (ex: each of the ends) is located at such pixel, co-ordinates outputs to determine each landmark location in x-y co-ordinates normalized form 0-1 and calculates the center of mass of each landmark, all collectively AI model outputs. A validity module is deployed at 316 and assigns a score relative to a selected threshold. At step 318, when a validity score is below a threshold for either landmark end, this is filtered our as low confidence, and no measurement is taken (even if one end returns as high confidence). In this case, the validity module directs the repeat of steps 310-316 as illustrated by the arrow and line back to step 310. Such redirection enables other images to be selected of the feature, and may allow better images to be processed, in order to acquire the desired measurement. Such new images may not require rescanning but could be selected from a cineloop and processed through steps 310-316. At step 320, when a validity score meets or exceeds a threshold for both landmark “ends”, it is accepted, and subsequently at step 322 a Euclidean distance between co-ordinates if calculated and at step 322, pixel distance between the landmark ends is converted to physical distance.

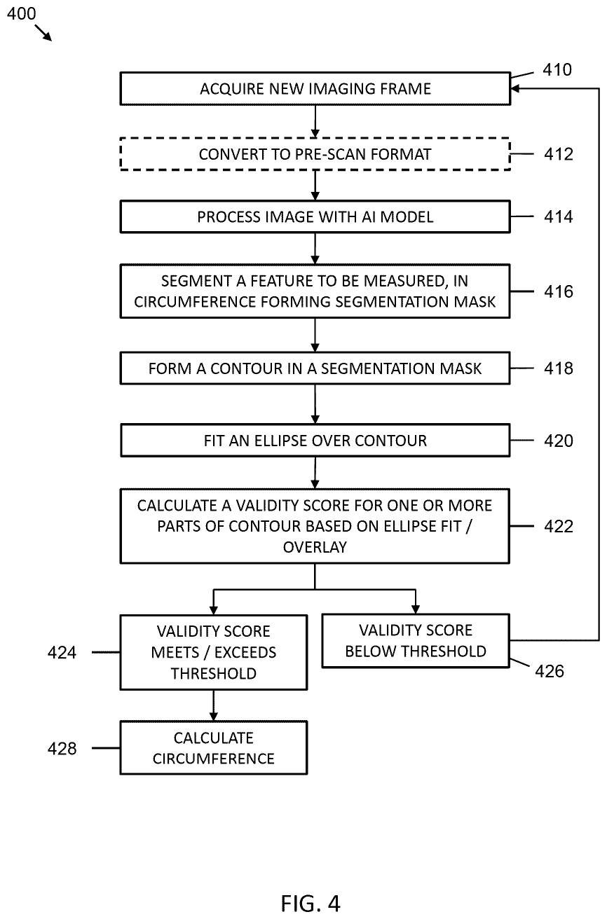

A flowchart diagram of a method in accordance with one embodiment of the invention, generally indicated at 400, is shown in FIG. 4. At step 410, a new ultrasound image is acquired. It is to be understood such new ultrasound image may be processed prior to display of an ultrasound image for viewing to a user, and, at step 412, the ultrasound image may be converted to pre-scan format for the purpose of processing through an AI model of the invention. The AI model, at step 414 engages in processing the image and at step 416 segments a feature to be measured from the ultrasound image, such feature comprising a circumference to be measured, such segmentation creating a segmentation mask on the feature. It is to be understood that, for example, in ultrasound scanning of a fetus, the term “feature” may comprise the entire fetus in early gestation and may comprise one or more parts thereof (for example, head and abdomen) in later gestation. The AI model forms a contour in the segmentation mask at step 418 and at step 420, an ellipse if fitted over the contour. At step 422, a validity module calculates a validity sore for one or more parts of the contour based on the ellipse fit (degree of fit and overlay) to the contour. At step 424, when a validity score meets or exceeds a threshold it is accepted, and subsequently at step 428 a circumference is calculated. When a validity score is below a threshold, as shown in step 426, this is filtered our as low confidence, and no circumferential measurement is taken. In this case, the validity module directs the repeat of steps 410-422 as illustrated by the arrow and line back to step 410. Such redirection enables other images to be selected of the feature, and may allow better images to be processed, in order to acquire the desired circumferential measurement with a higher degree of confidence. Such new images may not require rescanning but could be selected from a cineloop and processed through steps 410-422.

FIG. 5 illustrates a system schematic and architecture generally shown as 500, processing an ultrasound image of a fetal head, 510 for the purpose of calculating HC and BPD. An AI model comprising a plurality of encoder layers (511-514) and decoder layers (515-517) segments the head resulting in: i) segmentation mask 518 which is processed through ellipse fitting at 521 and circumference measurement as shown; ii) BPD outer/inner heatmaps (519 and 520) which are passed through DSNT layers 522 to produce coordinates 523, pairs of which form the associated BPD distance measurement. Validities are calculated for both types and are used to reject low confidence samples, in accordance with the methods described herein.

FIG. 6 shows four schematics, at 600 of exemplary B-Mode images with GT labels. From left to right: 1) ultrasound image 610 comprising fetus segmentation 618 with CRL head 622 and rump 620 landmarks; 2) ultrasound image 612 comprising head segmentation 624 with BPD inner 626 and outer 628 landmarks; 3) ultrasound image 614 comprising femur segmentation 634 with FL distal 630 and proximal 632 landmarks, and femur between landmarks shown as 634; and 4) ultrasound image 616 comprising abdomen segmentation 638 (no associated landmarks).

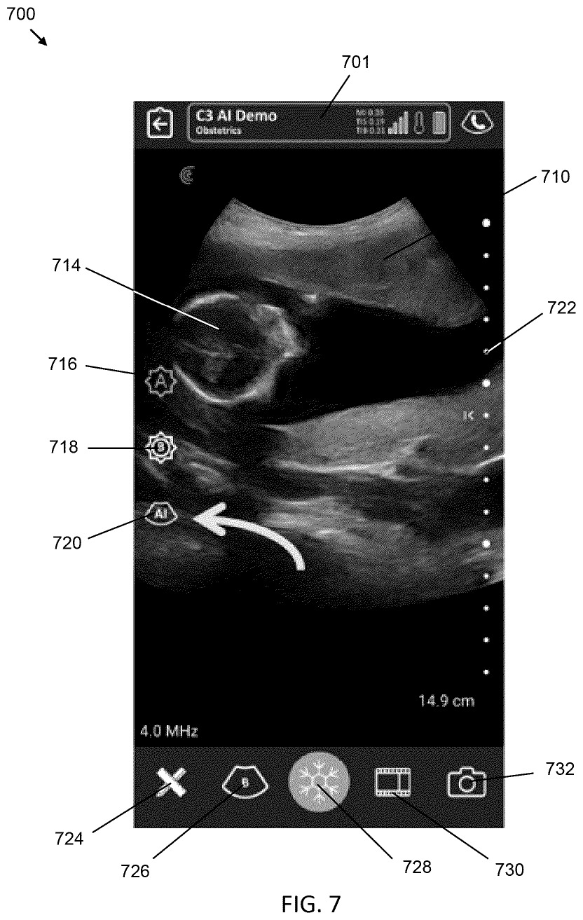

FIGS. 7-17 are schematics of exemplary user interface displays, each setting out aspects of the method, in accordance with various aspects of the invention, for initiating the deployment of both the trained AI model and the validity/measurement module, shown generally at 700, 800, 900, 1000, 1100, 1200A/B, 1300A/B, 1400A/B, 1500A/B, 1600, and 1700. In FIG. 7, at interface screen 701, there is displayed image 710 comprising fetus 714. A user is in an OB preset for each of these steps in FIGS. 7-17. A selection of icons at the left of interface screen 701 includes an AI icon 720, which, once selected, activates detection and highlighting processes. The AI icon is further highlighted by the yellow arrow. Additional icons include image appearance icon at 718 and overall gain icon at 716. Interface screen 701 additionally shows displayed image 710 comprising fetus 714. Interface screen 701 may additionally comprise one or more controls and guides including but not limited to: imaging mode selector 713, freeze button 728, video icon 730, screen capture icon 732, tools icon 724 and depth indicator 722.

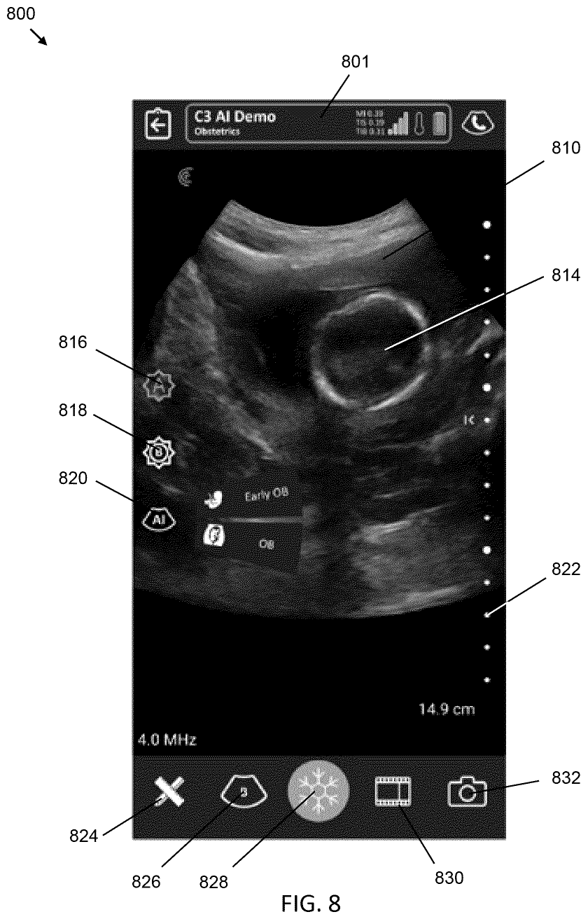

In FIG. 8, at interface screen 801, there is displayed image 810 comprising fetus 814. A selection of icons at the left of interface screen 801 includes an AI icon 820, which, once selected, activates detection and highlighting processes. The AI icon is further highlighted by the yellow arrow. Additional icons include image appearance icon at 818 and overall gain icon at 816. Interface screen 801 additionally shows displayed image 810 comprising fetus 814. Interface screen 801 may additionally comprise one or more controls and guides including but not limited to: imaging mode selector 813, freeze button 828, video icon 830, screen capture icon 832, tools icon 824 and depth indicator 822. A circular carousel openable from AI icon 820 is viewable with all available AI models from which a user may make a selection. To use OB AI, a user may select either “OB” or “Early OB” as shown. “Early OB” may be selected for CRL measurement when gestational age is <13 weeks, whereas “OB” may be selected for BPD, HC, AC, or FL measurements when gestational age is >13 weeks. Once AI is activated, AI icon button may change colour and/or offer a visual “on” indicator.

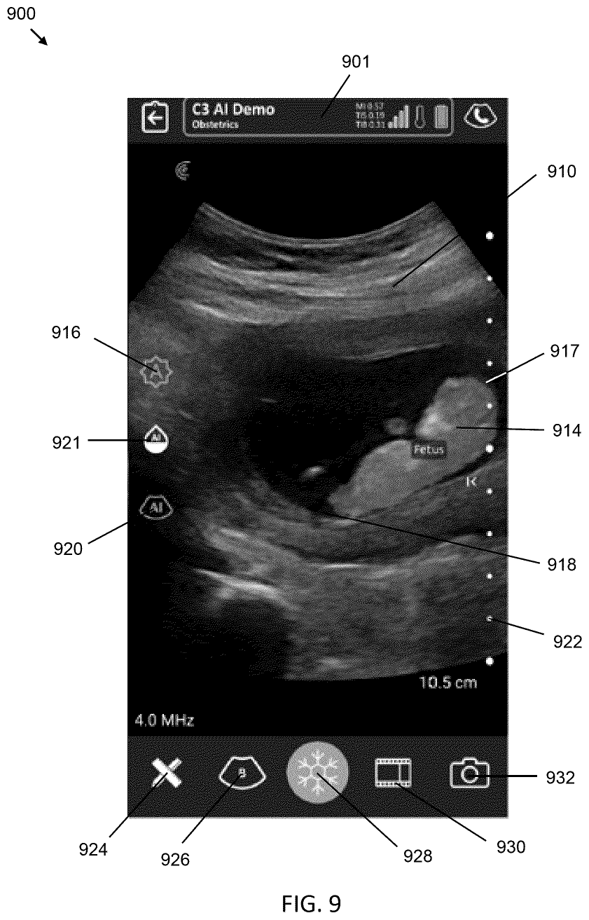

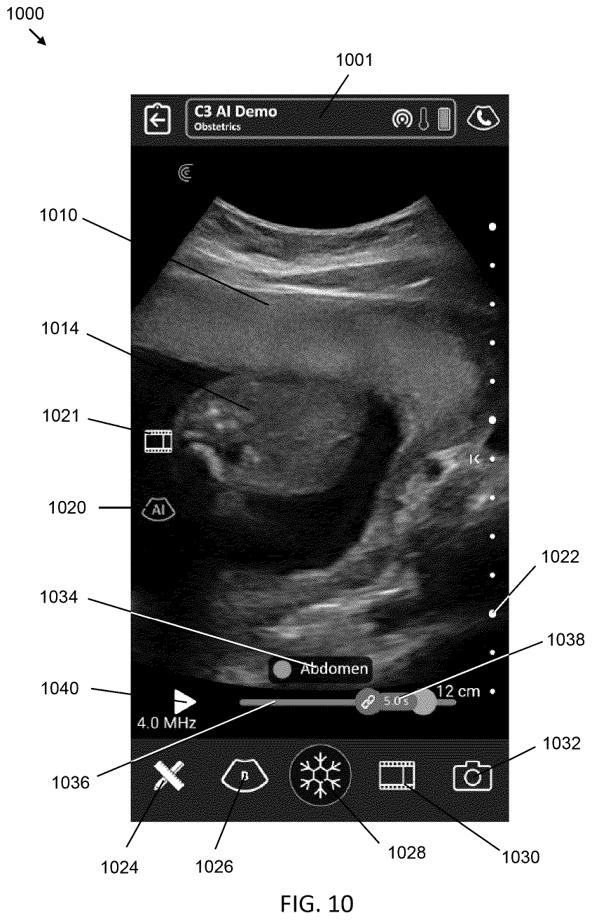

Once OB AI is activated, the AI icon may turn color and the desired fetal anatomy when in view is automatically highlighted by a colored mask (here green) and as shown in FIGS. 9 and 10. The fetus is highlighted in a case of <13 weeks and a secondary parietal feature (ex: the abdomen and/or head) is highlighted in a case of >13 weeks. In FIG. 9, at interface screen 901, there is displayed image 910 comprising early gestational fetus 914 with colored mask. AI icon 920 may be highlighted and additional icons include image appearance icon at 918 and overall gain icon at 916. Color markers are shown for identified crown 917 and rump 918. Interface screen 901 may additionally comprise one or more controls and guides including but not limited to: imaging mode selector 913, freeze button 928, video icon 930, screen capture icon 932, tools icon 924 and depth indicator 922.

In FIG. 10, at interface screen 1001, there is displayed image 1010 comprising mid to late gestational fetus 1014 with colored (segmentation) mask on abdomen (that being the only part of fetus in view in the ultrasound image). AI icon 1020 may be highlighted and additional icons include image appearance icon at 1018 and overall gain icon at 1016. Interface screen 1001 may additionally comprise one or more controls and guides including but not limited to: freeze button 1028, video icon 1030, screen capture icon 1032, tools icon 1024 and depth indicator 1022. Slider icon/cineloop play arrow 1040 enables user directed playback and scrolling of cineloop across time bar 1036 (with timestamp 1038). An information bar at the top of screen interface may comprise status information including type of scanner in use, battery levels, and other information pertinent to user.

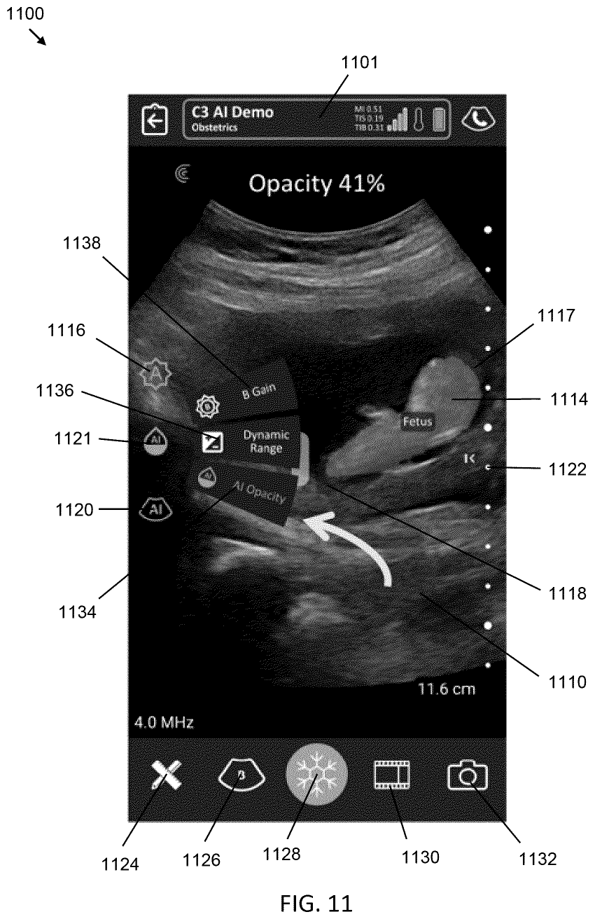

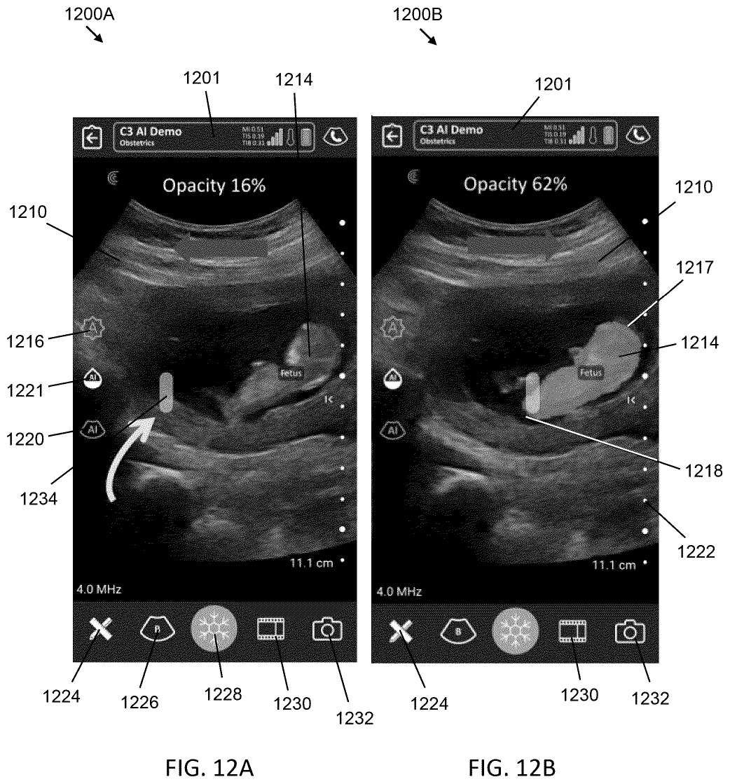

The opacity of a segmentation mask can also be adjusted as shown generally as 1100, 1200A and 1200B, in FIGS. 11, 12A and 12B. In FIG. 11, at interface screen 1101, there is displayed image 1110 comprising early gestational fetus 1114 with colored mask. AI icon 1120 may be highlighted and additional icons include image appearance icon at 1118 and overall gain icon at 1116. Color markers are shown for identified crown 1117 and rump 1118. Interface screen 1101 may additionally comprise one or more controls and guides including but not limited to: imaging mode selector 1113, freeze button 1128, video icon 1130, screen capture icon 1132, tools icon 1124 and depth indicator 1122. Activation of slider control 1121 exposes a carousel wheel exposing icons for AI opacity 1134, dynamic range 1118 and B gain 1138. Adjustments to opacity are enabled through user activation of the AI opacity icon 1134 and interface sliding scale adjustments as depicted in FIGS. 12A and 12B wherein the opacity of the segmentation mask can be adjusted by dragging the slider control horizontally. Dragging it to the left will reduce the opacity of the segmentation mask, while dragging it to the right will increase the opacity of the mask. FIGS. 12A and 12B illustrate displayed ultrasound image 1210 comprising segmented fetus 1214 with color markers for identified crown 1217 and rump 1218. Scroll directions are highlighted by the arrows (opacity changing from 41% n FIG. 11, 16% in FIG. 12A to 62% in FIG. 12B). Opacity is displayed on the images upon change. Interface screen 1201 may additionally comprise one or more controls and guides including but not limited to: freeze button 1228, video icon 1230, screen capture icon 1232, tools icon 1224 and depth indicator 1222.

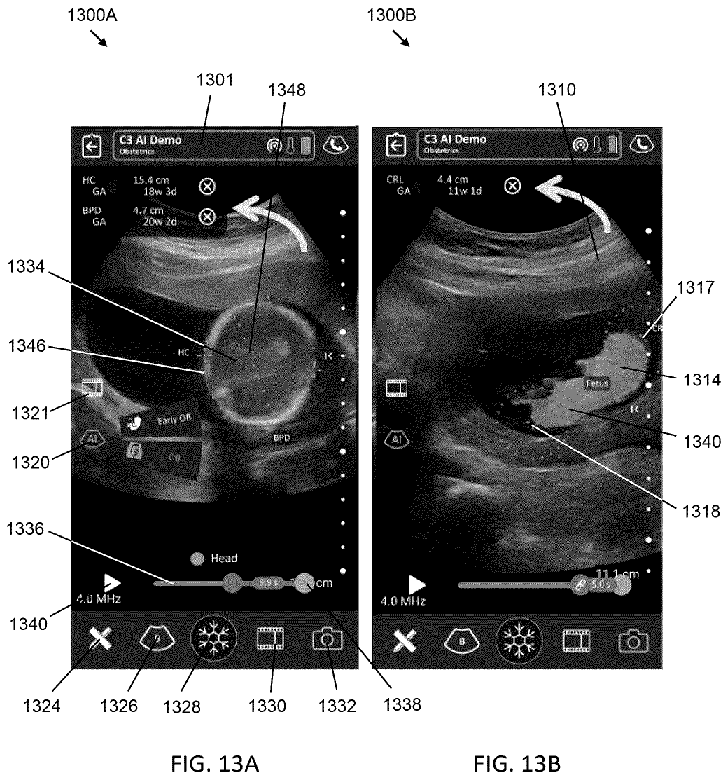

In an embodiment of the invention, to display the acquired measurements and corresponding gestational age, as calculated using one or more the validated measurements, a user pauses the acquisition of ultrasound images by pressing the freeze button (shown as 1328 in FIGS. 13A and 13B). Calipers will be placed automatically, with measurements calculated automatically and displayed in the top left corner of the interface screen as shown in FIGS. 13A and 13B. More specifically, in FIG. 13A, at interface screen 1301, there is displayed image 1310 comprising fetal head 1334. Two measurements are calculated and displayed at the top left corner of the screen: HC (1346) and BPD (1348). HC is acquired by the processing of ultrasound image using the AI model of the invention, to segment by identifying the fetus (in whole or part) and fetal anatomies in an ultrasound image; and identifying pixels belonging to the fetus and labelling them as 1s and the rest of the pixels as 0s and thereafter fitting an ellipse over the segmented parietal structure to predict a validity score, which is thereafter accepted or rejected based upon a threshold. In FIG. 13A, a gestational age based upon the HC of 15.4 cm is noted at 18 weeks and 3 days. BPD is acquired by the processing of ultrasound image using the AI model of the invention to identify and verify/validate landmark points, as described herein. In FIG. 13A, a gestational age based upon the BPD of 4.7 cm is noted at 20 weeks and 2 days. Interface screen 1301 may additionally comprise one or more controls and guides including but not limited to: freeze button 1328, video icon 1330, screen capture icon 1332, tools icon 1324 and depth indicator 1322.

In FIG. 13B, at interface screen 1301, there is displayed image 1310 comprising an early gestational fetus 1314. One measurement is calculated and displayed at the top left corner of the screen: CRL (1340). CRL is acquired by the processing of ultrasound image using the AI model of the invention, wherein a DSNT layer transforms spatial heatmaps, identified using the AI model, into numerical coordinates. For a landmark output, a 2D Gaussian heatmap is generated, centered around a labelled coordinate location the landmark validity as the intensity of the heatmap pixels surrounding the predicted coordinate. For each of the two landmarks (in FIG. 13B crown and rump), if both landmarks are valid, the Euclidean distance between the coordinates (in pixels) is calculated and thereafter the pixel-distance to physical distance across CRL 1340 (in mm) is calculated using pixel spacing information from the probe's metadata. Segmented fetus 1314 shows color markers for identified crown 1317 and rump 1318. Should there be no color at either of the landmarks, this is a visual cue to a user that such a landmark was not validated for measurement.

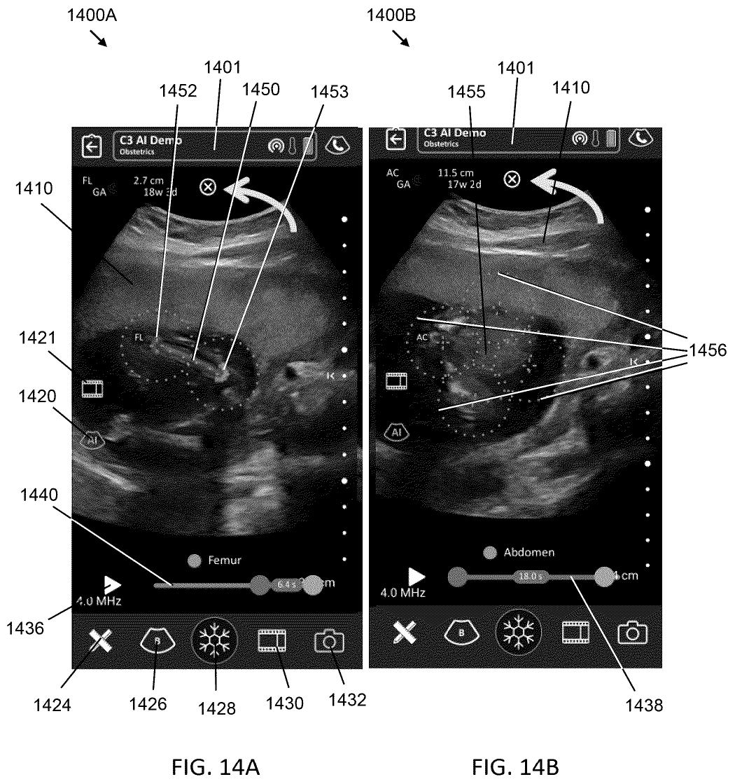

In a further embodiment of the invention, to display the acquired measurements and corresponding gestational age, as calculated using one or more the validated measurements, a user pauses the acquisition of ultrasound images by pressing the freeze button (shown as 1428 in FIGS. 14A and 14B). Measurements calculated automatically and displayed in the top left corner of the interface screen. In FIG. 14A, at interface screen, there is displayed image 1410 comprising a femur 1450. FL is calculated and displayed at the top left corner of the screen: FL (1450) of 2.7 cm with predicted gestational age of 18 weeks 3 days. FL is acquired by the processing of ultrasound image using the AI model of the invention, wherein a DSNT layer transforms spatial heatmaps, identified using the AI model, into numerical coordinates. For each landmark output, a 2D Gaussian heatmap is generated, centered around a labelled coordinate location the landmark validity as the intensity of the heatmap pixels surrounding the predicted coordinate. For each of the two landmarks (in FIG. 14A as distal end 1453 in blue and proximal end 1452 in pink ends of femur), if both distal and proximal marks are valid, the Euclidean distance between the coordinates (in pixels) is calculated and thereafter the pixel-distance to physical distance across CRL 1340 (in mm) is calculated using pixel spacing information from the probe's metadata. Should there be no color at either of the proximal and distal landmarks, this is a visual cue to a user that such a landmark was not validated for measurement. Interface screen 1401 may additionally comprise one or more controls and guides including but not limited to: freeze button 1428, video icon 1430, screen capture icon 1432, tools icon 1424 and depth indicator 1422. Slider icon/cineloop play arrow 1440 enables user directed playback and scrolling of cineloop across time bar 1436 (with timestamp 1438.

In FIG. 14B, there is displayed image 1410 comprising fetal abdomen 1455. One measurement is calculated and displayed at the top left corner of the screen: AC (1455). AC is acquired by the processing of ultrasound image using the AI model of the invention, to segment by identifying the fetus (in whole or part, here, the abdomen) and fetal anatomies in an ultrasound image; and identifying pixels belonging to the part of the fetus (here the abdomen) and labelling them as 1s and the rest of the pixels as 0s and thereafter fitting an ellipse over the segmented parietal structure to predict a validity score, which is thereafter accepted or rejected based upon a threshold. In FIG. 14B, a gestational age based upon the AC of 11.5 cm is noted at 17 weeks and 2 days.

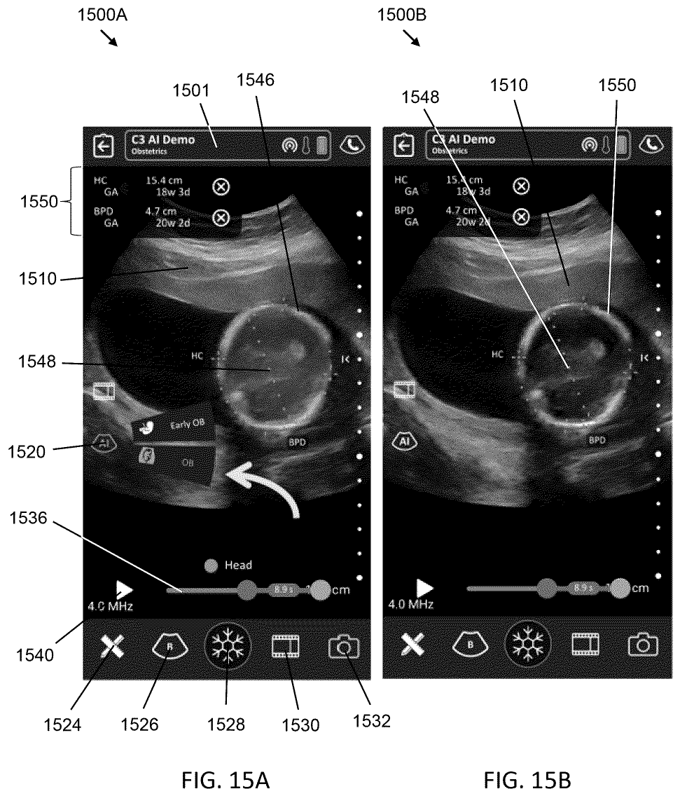

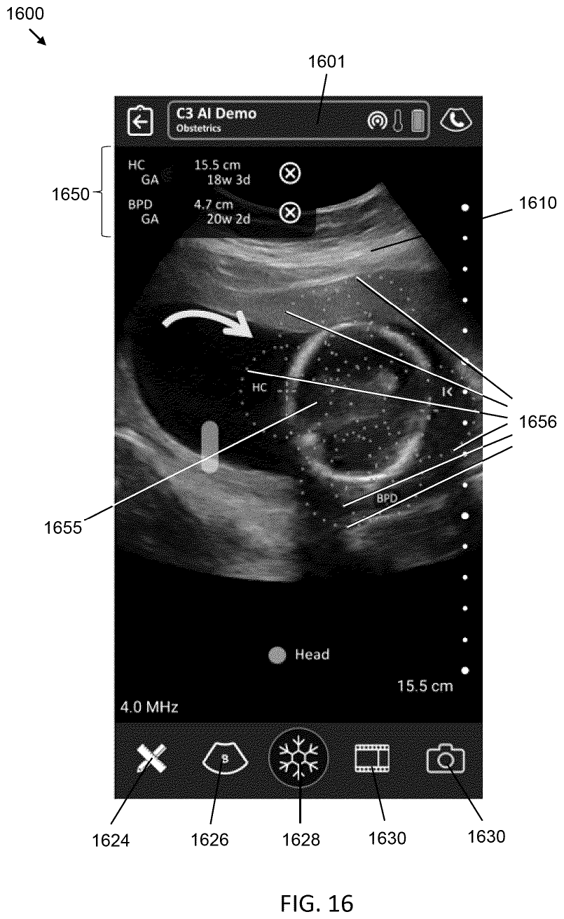

FIGS. 15A and 15B illustrate the removal of the segmentation mask and disabling of the OB AI preset. A user may tap the AI icon 1520 and de-select the chosen preset (“OB” or “Early OB”) from the AI carousel wheel. AI icon 1520 may change color or highlighting (ex: turn white). By way of example, an orange-coloured AI button may signify that OB AI is on, whereas a white coloured AI button may signify that OB AI is off. A segmentation mask can also be disabled in freeze mode. Once image acquisition is frozen, a user may disable AI by tapping AI icon 1520. This is particularly useful for those users who want to see the ultrasound image without the segmentation mask overlay. For this option, refer to FIGS. 15A and 15B and 16.

FIG. 15A shows a segmentation mask (shown in green) overlayed on fetal head. FIG. 15A, at interface screen 1501, there is displayed image 1510 comprising fetal head 1546 Two measurements are calculated and displayed at 1551 the top left corner of the screen: HC (1546) and BPD (1548). In FIG. 15A, a gestational age based upon the HC of 15.4 cm is noted at 18 weeks and 3 days. BPD is acquired by the processing of ultrasound image using the AI model of the invention to identify and verify/validate landmark points, as described herein. In FIG. 15A, a gestational age based upon the BPD of 4.7 cm is noted at 20 weeks and 2 days. Interface screen 1501 may additionally comprise one or more controls and guides including but not limited to: freeze button 1528, video icon 1530, screen capture icon 1532, tools icon 1524 and depth indicator 1522. At FIG. 15B, the segmentation mask is removed from the image shown in 15A. In this way, caliper points and other measurement points are viewable in the absence of a mask.

Similarity, in FIG. 16, an ultrasound image comprising a fetal head is shown, like that in FIG. 13A but without the segmentation mask overlay. Thus, at interface screen 1601, there is displayed image 1610 comprising fetal head 1655. Two measurements are calculated and displayed at the top left corner of the screen: HC and BPD. In FIG. 16, a gestational age based upon the HC of 15.4 cm is noted at 18 weeks and 3 days. BPD is acquired by the processing of ultrasound image using the AI model of the invention to identify and verify/validate landmark points, as described herein. In FIG. 16, a gestational age based upon the BPD of 4.7 cm is noted at 20 weeks and 2 days. Interface screen 1601 may additionally comprise one or more controls and guides including but not limited to: freeze button 1628, video icon 1630, screen capture icon 1632, tools icon 1624 and depth indicator 1622. With the segmentation mask removed from the image shown in FIG. 16, demarcation and caliper points around 1656 and other measurement points are more clearly viewable, as might be a preference of the user.

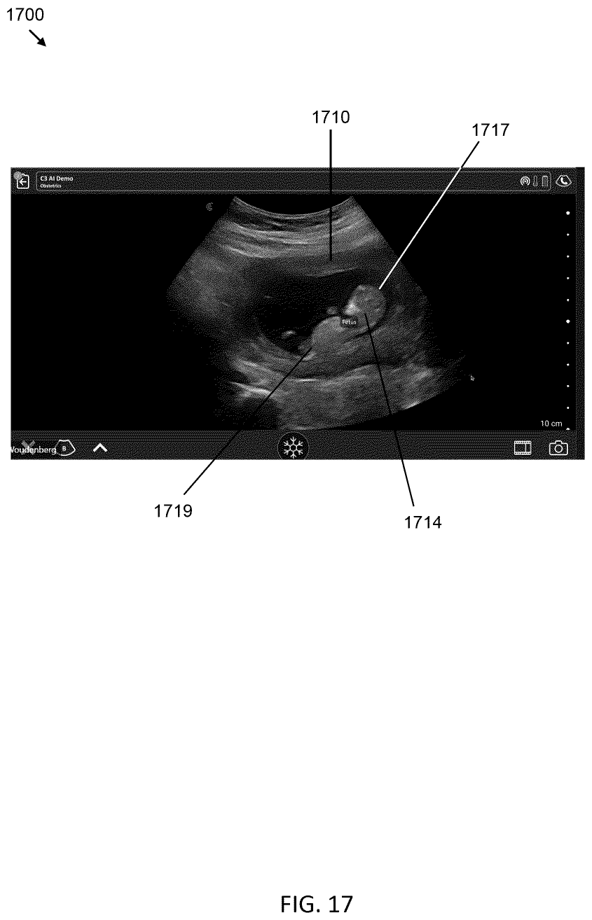

FIG. 17 is illustrative of a display in which one or more defined values fail validation, and hence no measurement would be displayed for a user as a quality control feature of the method and system of the invention. Segmented fetus 1714 (shown in green mask overlay) shows a color marker for identified crown 1317 (confirming validated landmark) but fails to apply color to rump 1719. As there is no color at the rump “landmark”, this is a visual cue to a user that such a landmark was not validated for measurement. Options for a user include acquiring additional ultrasound images and/or scrolling through cineloop images and reapplying the method of the invention (for example the steps of one or more of FIGS. 1-4) and/or adjusting a threshold.

As described herein, in the deployment of the AI model of the invention, identification and prediction may be achieved by a variety of methods, including, but not limited to, segmentation of boundaries/edge detection, contouring and classification. This invention is not intended to be limited to any one mode of AI-model-deployment. The product of the AI model is one or more outputs as described herein. The AI model output is automatically conveyed to a validation/measurement module which makes processing decisions as to the acquisition or non-acquisition of measurements and further calculation based on those measurements.

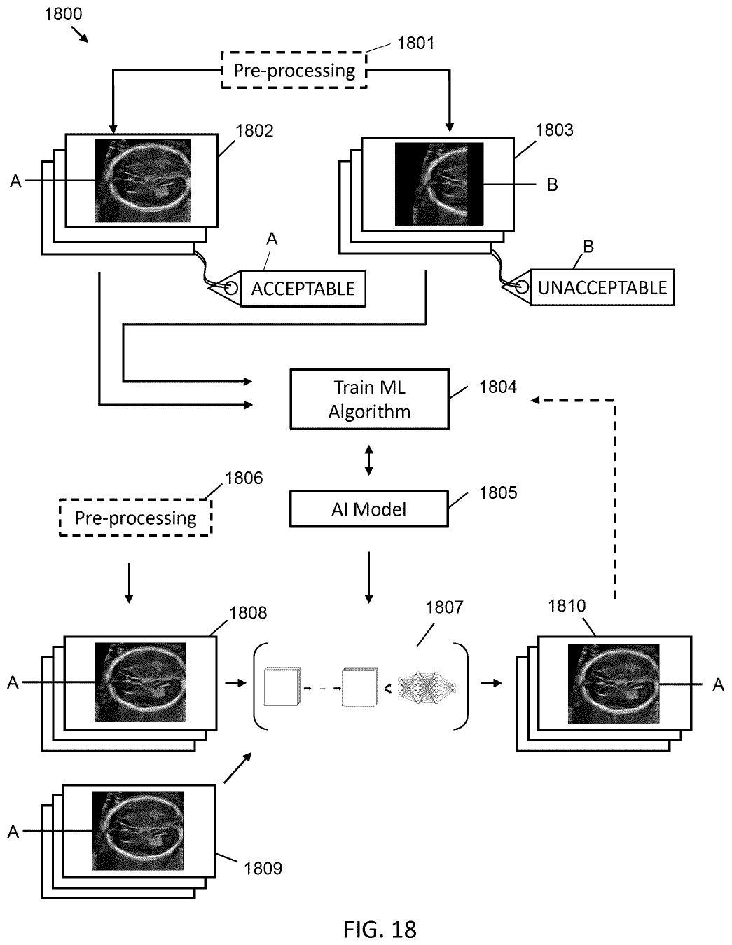

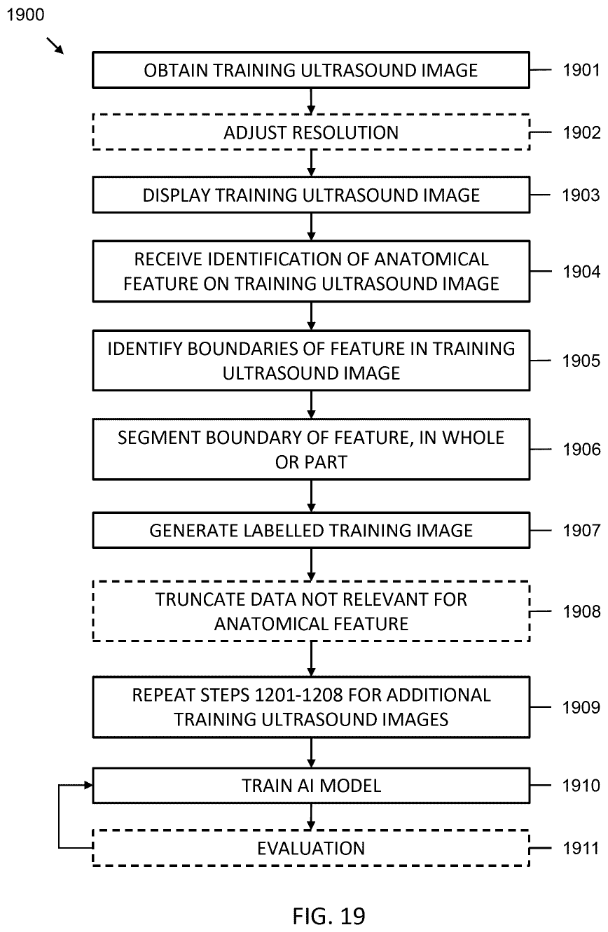

In various embodiments, a variety of means to segment an ultrasound image may be used. For example, segmentation may be performed by dividing it into multiple parts or regions that belong to the same class. This task of clustering is based on specific criteria, for example, color or texture and is referred to as pixel-level classification. This involves partitioning images into multiple segments or objects using techniques including, but not limited to 1) thresholding, wherein a threshold value is set, and all pixels with intensity values above or below the threshold are assigned to separate regions; 2) region growing, wherein an ultrasound image is divided into several regions based on similarity criteria. This segmentation technique starts from a seed point and grows the region by adding neighboring pixels with similar characteristics; 3) edge-based segmentation wherein segmentation techniques are based on detecting edges in the ultrasound image and these edges represent boundaries between different regions that are detected using edge detection algorithms; 4) clustering, wherein groups of pixels are clustered based on similarity criteria. These criteria can be color, intensity, texture, or any other feature; 5) active contours, also known as snakes, wherein curves that deform are used to find the boundary of an object in an image. These curves are controlled by an energy function that minimizes the distance between the curve and the object boundary; 6) deep learning-based segmentation, such as by employing Convolutional Neural Networks (CNNs), which employ a hierarchical approach to image processing, where multiple layers of filters are applied to the input image to extract high-level features, the training of which is described herein in FIGS. 18 and 19.

Referring to FIG. 18, shown there generally at 1800 is a schematic diagram of a training and deployment of an AI model 1805. According to an embodiment of the present invention, there is shown a method of training a neural network 1807 to so that when the AI model is deployed, a computing device identifies a feature to be measured, in whole or part and selects a definable value of the feature.

For training, a number of ultrasound frames of a ROI (in whole view, from varying perspectives and parts thereof) may be acquired using an ultrasound scanner (hereinafter “scanner”, “probe”, or “transducer” for brevity). The ultrasound frames may be acquired by fanning a series of a planes (with a frame each containing a sequence of transmitted and received ultrasound signals), through an angle and capturing a different ultrasound frame at each of a number of different angles. During the scanning, the scanner may be held steady by an operator of the scanner while a motor in the head of the scanner tilts the ultrasonic transducer to acquire ultrasound frames at different angles. Additionally, or alternatively, other methods of acquiring a series of ultrasound frames may be employed, such as using a motor to translate (e.g., slide) the ultrasonic transducer or rotate it, or manually tilting, translating or rotating the ultrasound scanner.

The AI model if preferably trained with a robust selection of images of varying views. For example, these different views may include transverse plane views of a ROI, including views from different angles that combine any of a sagittal plane view, a coronal plane view, or a transverse plane view. In these embodiments, the scanner may be placed in an arbitrary orientation with respect to the ROI, provided that the scanner captures at least a portion of the ROI.

In some embodiments, ultrasound scans of a ROI, for training, may be acquired from medical examinations. During the scans, images may be obtained; however, for training of the AI model of the invention, non-clinically useful or acceptable images may also be used.

Referring still to FIG. 11, training ultrasound frames (1802 and 1803) may include ultrasound frames with features that are tagged as acceptable (A) and representative of images which are segmented, identified, classified or otherwise meet the requirements of the AI model or alternatively are tagged respectively as unacceptable (B) and unrepresentative of such division, classification and/or segmentation. By way of example, in ultrasound frame 1802, which is marked as acceptable, there is provided an image which is marked as correctly and at least adequality segmented and identified. Conversely, ultrasound frame 1803, of the same ROI as ultrasound frame 1802, is marked as unacceptable, due to the fact that the features are unclear, and/or unclear and/or are at least non-adequality segmented.

Both the training ultrasound frames labeled as Acceptable and Unacceptable, for each particular ROI (whole or part), may themselves be used for training and/or reinforcing AI model 1805. This is shown in FIG. 18 with tracking lines from both 1811 to training algorithm step 1810. As such, ultrasound frame 1803 may be employed for training as an unacceptable image.

In some embodiments, an optional pre-processing act 1801 may be performed on the underlying ultrasound image frames 1802 and 1803 to facilitate improved performance and/or accuracy when training the machine learning (ML) algorithm. For example, it may be possible to pre-process the ultrasound images 1802 and 1803 through a high contrast filter to reduce the granularity of greyscale on the ultrasound images 1802 and 1803.

Additionally, or alternatively, it may be possible to reduce scale of the ultrasound images 1802 and 1803 prior to providing the ultrasound images 1802 and 1803 to the training algorithm step 1804. Reducing the scale of ultrasound images 1802 and 1803 as a preprocessing step may reduce the amount of image data to be processed during the training act 1804, and thus may reduce the corresponding computing resources required for the training act 1804 and/or improve the speed of the training act 1804.

Various additional or alternative pre-processing acts may be performed in act 1801. For example, these acts may include data normalization to ensure that the various ultrasound frames 1802 and 1803 used for training have generally the same dimensions and parameters.

Referring still to FIG. 18, the various training frames 1802 and 1803 may, at act 1804, be used to train a ML algorithm. For example, the various training ultrasound frames 1802 and 1103, may be inputted into deep neural network 1807 that can learn how to predict boundaries of features in new ultrasound images as compared to all trained and stored images.

The result of the training may be the AI model 1805, which represents the mathematical values, weights and/or parameters learned by the deep neural network to predict segmented boundaries of features, within a ROI, in whole or part. The training act 1804 may involve various additional acts (not shown) to generate a suitable AI model 1805. For example, these various deep learning techniques such as regression, classification, feature extraction, and the like. Any generated AI models may be iteratively tested to ensure they are not overfitted and sufficiently generalized for creating the comparison and list of probabilities in accordance with method of the invention.

In some embodiments, using a cross-validation method on the training process would optimize neural network hyper-parameters to try to ensure that the neural network can sufficiently learn the distribution of all possible image types without overfitting to the training data. In some embodiments, after finalizing the neural network architecture, the neural network may be trained on all of the data available in the training image files.

In various embodiments, batch training may be used, and each batch may consist of multiple images, thirty-two for example, wherein each example image may be gray-scale, preferably 128*128 pixels although 256*256 pixels and other scaled may be used, without any preprocessing applied to it.

In some embodiments, the deep neural network parameters may be optimized using the Adam optimizer with hyper-parameters as suggested by Kingma, D. P., Ba, J. L.: Adam: a Method for Stochastic Optimization, International Conference on Learning Representations 2015 pp. 1-15 (2015), the entire contents of which are incorporated herewith. The weight of the convolutional layers may be initialized randomly from a zero-mean Gaussian distribution. In some embodiments, the Keras™ deep learning library with TensorFlow™ backend may be used to train and test the models.

In some embodiments, during training, many steps may be taken to stabilize learning and prevent the model from over-fitting. Using the regularization method, e.g., adding a penalty term to the loss function, has made it possible to prevent the coefficients or weights from getting too large. Another method to tackle the over-fitting problem is dropout. Dropout layers limit the co-adaptation of the feature extracting blocks by removing some random units from the neurons in the previous layer of the neural network based on the probability parameter of the dropout layer. Moreover, this approach forces the neurons to follow overall behaviour. This implies that removing the units would result in a change in the neural network architecture in each training step. In other words, a dropout layer performs similar to adding random noise to hidden layers of the model. A dropout layer with the dropout probability of 0.5 may be used after the pooling layers.

Data augmentation is another approach to prevent over-fitting and add more transitional invariance to the model. Therefore, in some embodiments, the training images may be augmented on-the-fly while training. In every mini-batch, each sample may be translated horizontally and vertically, rotated and/or zoomed, for example. The present invention is not intended to be limited to any one particular form of data augmentation, in training the AI model. As such, any mode of data augmentation which enhances the size and quality of the data set and applies random transformations which do not change the appropriateness of the label assignments may be employed, including but not limited to image flipping, rotation, translations, zooming, skewing, and elastic deformations.

Referring still to FIG. 18, after training has been completed, the sets of parameters stored in the storage memory may represent a trained neural network of a plurality of images of ROIs which identifies and segments boundaries of features with each ROI, in whole or part.

In order to assess the performance of AI model 1805, the stored model parameter values can be retrieved any time to perform image assessment through applying an image to the neural networks (shown as 1807) represented thereby. In some embodiments, the deep neural network may include various layers such as convolutional layers, pooling layers, and fully connected layers. In some embodiments, the final layers may include a softmax layer as an output layer having outputs which eventually would demonstrate respective determinations that an input set of pixels fall within a particular area above or below a feature boundary, in the training images. Accordingly, in some embodiments, the neural network may take at least one image as an input and output a binary mask indicating which pixels belong to the area above a feature boundary (or part thereof), e.g., the AI model classifies which area each pixel belongs to.

To increase the robustness of the AI model 1805, in some embodiments, a broad set of training data may be used at act 1804. For example, it is desired that ultrasound images of a plurality of different ROIs, across a plurality of anatomical regions in a body, in whole and a variety of parts thereof, from views including but not limited to coronal and/or transverse plane views, including views from different angles that combine any of a sagittal plane view, a coronal plane view, or a transverse plane view.

More specifically, training images 1802 and 1803 may be labeled with one or more features associated with/are hallmarks of a particular ROI, including key anatomical features therein. This may include identifying a variety of features visualized in the captured training image. In at least some embodiments, this data may be received from trainer/user input. For example, a trainer/user may label the features relevant for the application visualized in each training image.

The image labeling can be performed, for example, by a trainer/user observing the training ultrasound images, via a display screen of a computing device, and manually annotating the image via a user interface. In some aspects, the training ultrasound images used for the method herein will only be images in which the image quality is of a sufficient quality threshold to allow for proper and accurate feature identification. For example, this can include training ultrasound images having a quality ranging from a minimum quality in which target features are just barely visible for labelling (e.g., annotating), to excellent quality images in which the target features are easily identifiable. In various embodiments, the training medical images can have different degrees of images brightness, speckle measurement and SNR. Accordingly, training ultrasound images 1102 and 1103 can include a graduation of training images ranging from images with just sufficient image quality to high image quality. In this manner, the machine learning model may be trained to identify features on training medical images that have varying levels of sufficient image quality for later interpretation and probability assessment.

Overall, the scope of the invention and accorded claims are not intended to be limited to any one particular process of training AI model 1805. Such examples are provided herein by way of example only. AI model 1805 may be trained by both supervised and unsupervised learning approaches although due to scalability, unsupervised learning approaches, which are well known in the art, are preferred. Other approaches may be employed to strengthen AI model 1805.

The image labelling can be performed, for example, by a trainer/user observing the training ultrasound images, via a display screen of a computing device, and manually annotating the image via a user interface. In some aspects, the training ultrasound images used for the method herein will only be images in which the image quality is of a sufficient quality threshold to allow for proper and accurate feature identification. For example, this can include training ultrasound images having a quality ranging from a minimum quality in which target features are just barely visible for labelling (e.g., annotating), to excellent quality images in which the target features are easily identifiable. In various embodiments, the training medical images can have different degrees of images brightness, speckle measurement and SNR. Accordingly, training ultrasound images can include a graduation of training medical images ranging from images with just sufficient image quality to high image quality. In this manner, the machine learning model may be trained to identify features on training medical images that have varying levels of sufficient image quality for later interpretation and probability assessment.

Turning back to FIG. 18, once a satisfactory AI model 1805 is generated, the AI model 1805 may be deployed for execution on a neural network 1807 to identify and segment boundaries of features, in whole or part, within a ROI. Notably, the neural network 1807 is shown in FIG. 18 for illustration as a convolution neural network—with various nodes in the input layer, hidden layers, and output layers. However, in various embodiments, different arrangements of the neural network 1807 may be possible.

In various embodiments, prior to being processed for analysis as described herein, training ultrasound image frames may optionally be pre-processed in a manner analogous to the pre-processing act 112 in FIG. 1. (e.g., processing through a high contrast filter and/or scaling), to facilitate and improve accuracy in identifying and selecting boundaries of features, in whole or part.

The training images file may include an image identifier field for storing a unique identifier for identifying an image included in the file, a segmentation mask field for storing an identifier for specifying the to-be-trimmed area, and an image data field for storing information representing the image.

Referring again to FIG. 18, once a satisfactory AI model 1805 is generated, the AI model 1105 may be deployed for execution on a neural network 1807, as described fully herein, on new ultrasound images 1808. Notably, the neural network 1807 is shown in FIG. 18 for illustration as a convolution neural network—with various nodes in the input layer, hidden layers, and output layers. However, in various embodiments, different arrangements of the neural network 1807 may be possible.

In various embodiments, prior to being processed for feature segmentation, the new ultrasound images 1808 may optionally be pre-processed. This is shown in FIG. 18 with the pre-processing act 1806 in dotted outline. In some embodiments, these pre-processing acts 1806 may be analogous to the pre-processing acts 1801 performed on the training ultrasound frames 1802 and 1803 (e.g., processing through a high contrast filter and/or scaling), to better align the new ultrasound images 1808 with the training ultrasound image frames, and thereby facilitate improved accuracy in feature segmentation. For example, pre-processing an input image may help standardize the input image so that it matches the format (e.g., having generally the same dimensions and parameters) of the training ultrasound images 1802 and 1803 that the AI model 1805 is trained on.

In various embodiments, the new ultrasound images 1808 may be live images acquired by an ultrasound imaging system (e.g., the system discussed with respect to FIGS. 20 and 21 below). For example, the AI model 1805 may be deployed for execution on the scanner 2031 and/or the display device 2050 discussed in more detail below. Additionally, or alternatively, the AI model 1805 may be executed on stored (as opposed to new) ultrasound images 1809 that were previously acquired (e.g., as may be stored on a Picturing Archiving and Communication System (PACS)).

Whether the images are stored ultrasound images 1809 or new ultrasound images 1808, the AI model 1805 enables the neural network 1807 to properly segment a feature within a ROI imaged in the new/stored ultrasound imaging data and created an identified and segmented image frame 1810.

FIG. 19 is flowchart diagram of the steps, generally indicated as 1900, for training the AI model of FIG. 18, according to an embodiment of the present invention. In some embodiments, method 1900 may be implemented as executable instructions in any appropriate combination of the imaging system 2030 (FIG. 20), for example, an external computing device connected to the imaging system 2030, in communication with the imaging system 2030, and so on. As one example, method 1900 may be implemented in non-transitory memory of a computing device, such as the controller (e.g., processor) of the imaging system 2030.

Referring still to FIG. 19, in step 1901, a training ultrasound image may be obtained. For example, a training ultrasound image may be acquired by the scanner 2031 (as shown in FIG. 20) transmitting and receiving ultrasound energy. The training ultrasound image may generally be a post-scan converted ultrasound image. While the method of FIG. 19 is described in relation to a single training ultrasound image, the method may also apply to the use of multiple training ultrasound images. While the method of FIG. 19 is described in relation to a post-scan ultrasound image, it is to be understood that pre-scan images, may be used, as described in U.S. patent application Ser. No. 17/187,851 filed Feb. 28, 2021, the entire contents of which are incorporated herein by reference.

Optionally, in step 1902 (as shown in dotted outline), the resolution of the training ultrasound image may be adjusted. For example, the resolution may be increased or decreased. The purpose of this may be to provide the labeler (e.g., a medical professional with relevant clinical expertise) with training ultrasound images that have a more standardized appearance. This may help to maintain a higher consistency with which the labeler identifies anatomical features in the training ultrasound images. Besides the resolution, other parameters of the training ultrasound image may also be adjusted such as input scaling, screen size, pixel size, aspect ratio, and the removal of dead space, as described above (including, for example, data augmentation and other preprocessing steps).

In step 1903, the training ultrasound image may be displayed on a display device, such as the display device 2050 discussed in more detail below in relation to FIG. 20. The labeler can then identify a particular anatomy in the training ultrasound image by, for example, tagging it with a name from a pull-down menu or by using other labeling techniques and modalities. The labeler then can mark the training ultrasound image around the particular anatomy that the labeler has identified in the training ultrasound image. In step 1904, the system that is used for the training may receive the identification of the anatomical feature(s) on the training ultrasound image. In step 1905, the system may generate, for example, from a labeler's marking inputs, identified boundaries of a feature or features in the training ultrasound frame. In step 1906, a boundary feature is segmented in order to, at step 1907, generate a labeled training image.

In various embodiments, steps may readily be interchanged with each other. For example, the generation of labeled confirmation at step 1907 may automatically proceed, without trainer intervention, using prior data which directs to the placement of feature boundaries.

Once the training ultrasound image has been segmented and labeled, the system may then remove, in step 1908, optionally, (as shown in dotted outline), regions of the labeled ultrasound data frame that are both outside the area of the identified boundary features and outside areas relevant for the AI model to recognize the particular anatomy within the ROI. For example, the labeled ultrasound data frame may be truncated at one or more sides. Truncation of some of the ultrasound data may allow the training of the AI model to proceed more quickly. There is provided a redirection at step 1909 to repeat steps 1901-1908 a plurality of times, for additional training images. At step 1910, AI model is trained. At step 1911, once training is completed, the AI model may be used to perform identifications and selections on an unseen dataset to validate its performance, such evaluation at step 1911 feeding data back to train the AI model at step 1910.

Referring to FIG. 20, an exemplary system 2030 is shown for automatically predicting one or more fetal biometric measurements on an ultrasound image comprising a fetus, in whole or part, and then automatically generating a prediction of fetal gestational age, based on the said one or more fetal biometric measurements. The system 2030 includes an ultrasound scanner 2031 with a processor 2032, which is connected to a non-transitory computer readable memory 2034 storing computer readable instructions 2036, which, when executed by the processor 2032, may cause the scanner 2031 to provide one or more of the functions of the system 2030. Such functions may be, for example, the acquisition of ultrasound data, the processing of ultrasound data, the scan conversion of ultrasound data, the transmission of ultrasound data or ultrasound frames to a display device 2050, the detection of operator inputs to the ultrasound scanner 2031, and/or the switching of the settings of the ultrasound scanner 2031.

Also stored in the computer readable memory 2034 may be computer readable data 2038, which may be used by the processor 2032 in conjunction with the computer readable instructions 2036 to provide the functions of the system 2030. Computer readable data 2038 may include, for example, configuration settings for the scanner 2031, such as presets that instruct the processor 2032 how to collect and process the ultrasound data for a plurality of ROIs and how to acquire a series of ultrasound frames. The scanner 2031 may include an ultrasonic transducer 2042 that transmits and receives ultrasound energy in order to acquire ultrasound frames. The scanner 2031 may include a communications module 2040 connected to the processor 2032. In the illustrated example, the communications module 2040 may wirelessly transmit signals to and receive signals from the display device 2050 along wireless communication link 2044. The protocol used for communications between the scanner 2031 and the display device 2050 may be WiFi™ or Bluetooth™, for example, or any other suitable two-way radio communications protocol. In some embodiments, the scanner 2031 may operate as a WiFi™ hotspot, for example. Communication link 2044 may use any suitable wireless communications network connection. In some embodiments, the communication link between the scanner 2031 and the display device 2050 may be wired. For example, the scanner 2031 may be attached to a cord that may be pluggable into a physical port of the display device 2050.

In various embodiments, the display device 2050 may be, for example, a laptop computer, a tablet computer, a desktop computer, a smart phone, a smart watch, spectacles with a built-in display, a television, a bespoke display or any other display device that is capable of being communicably connected to the scanner 2031. The display device 2050 may host a screen 2052 and may include a processor 2054, which may be connected to a non-transitory computer readable memory 2056 storing computer readable instructions 2058, which, when executed by the processor 2054, cause the display device 2050 to provide one or more of the functions of the system 2030. Such functions may be, for example, the receiving of ultrasound data that may or may not be pre-processed; scan conversion of received ultrasound data into an ultrasound image; processing of ultrasound data in image data frames; the display of a user interface; the control of the scanner 2031; the display of an ultrasound image on the screen 2052; the processing of new or stored ultrasound images against the AI model, the processing of AI model outputs in a validation and measurement module, and/or the storage, application, reinforcing and/or training of AI model 2005. The screen 2052 may comprise a touch-sensitive display (e.g., touchscreen) that can detect a presence of a touch from the operator on screen 2052 and can also identify a location of the touch in screen 2052. The touch may be applied by, for example, at least one of an individual's hand, glove, stylus, or the like. As such, the touch-sensitive display may be used for example to toggle text or to provide other inputs regarding the measurements and calculated volume. The screen 2052 and/or any other user interface may also communicate audibly. The display device 2050 is configured to present information to the operator during or after the imaging or data acquiring session. The information presented may include ultrasound images (e.g., one or more 2D frames), graphical elements, measurement graphics of the displayed images, user-selectable elements, user settings, and other information (e.g., administrative information, personal information of the patient, and the like).

Also stored in the computer readable memory 2056 may be computer readable data 2060, which may be used by the processor 2054 in conjunction with the computer readable instructions 2058 to provide the functions of the system 2030. Computer readable data 2060 may include, for example, settings for the scanner 2031, such as presets for acquiring ultrasound data; settings for a user interface displayed on the screen 2052; and/or data for one or more AI models and validity/measurement modules within the scope of the invention. Settings may also include any other data that is specific to the way that the scanner 2031 operates or that the display device 2050 operates. It can therefore be understood that the computer readable instructions and data used for controlling the system 2030 may be located either in the computer readable memory 2034 of the scanner 2031, the computer readable memory 2056 of the display device 2050, and/or both the computer readable memories 2034, 2056.

The display device 2050 may also include a communications module 2062 connected to the processor 2054 for facilitating communication with the scanner 2031. In the illustrated example, the communications module 2062 wirelessly transmits signals to and receives signals from the scanner 2031 on wireless communication link 2044. However, as noted, in some embodiments, the connection between scanner 2031 and display device 2050 may be wired.

Referring to FIG. 21, a system 2100 is shown in which there are multiple similar or different scanners 2101, 2102, 2104 connected to their corresponding display devices 2150, 2106, 2108 and either connected directly, or indirectly via the display devices, to a communications network 2110, such as the internet. The scanners 2131, 2102, 2104 may be connected onwards via the communications network 2110 to a server 2120. The server 2120 may include a processor 2122, which may be connected to a non-transitory computer readable memory 2124 storing computer readable instructions 2126, which, when executed by the processor 2122, cause the server 2120 to provide one or more of the functions of the system 2100. Such functions may be, for example, the receiving of ultrasound frames, the processing of ultrasound data in ultrasound frames, the control of the scanners 2131, 2102, 2104, the processing of using the AI model of new ultrasound images as described herein, creating one or more AI model outputs which are used to determine a validity score for the one or more definable values based on a threshold and/or machine learning activities related to one or more AI models 2105 (as discussed above in relation to the methods shown in FIGS. 1-4).

Also stored in the computer readable memory 2124 may be computer readable data 2128, which may be used by the processor 2122 in conjunction with the computer readable instructions 2126 to provide the functions of the system 2100. Computer readable data 2128 may include, for example, settings for the scanners 2131, 2102, 2104 such as preset parameters for acquiring ultrasound data, settings for user interfaces displayed on the display devices 2150, 2106, 2108, and data for one or more AI models 2105. Settings may also include any other data that is specific to the way that the scanners 2131, 2102, 2104 operate or that the display devices 2150, 2106, 2108 operate.

It can therefore be understood that the computer readable instructions and data used for controlling the system 2100 may be located either in the computer readable memory of the scanners 2131, 2102, 2104, the computer readable memory of the display devices 2150, 2106, 2108, the computer readable memory 2124 of the server 2120, or any combination of the foregoing locations.

As noted above, even though the scanners 2131, 2102, 2104 may be different, each ultrasound frame acquired may be used by the AI model 2105 for training purposes. Likewise, ultrasound frames acquired by the individual scanners 2131, 2102, 2104 may all be processed against the AI model 2105 for reinforcement of the AI model 2105. In some embodiments, the AI models 2105 present in the display devices 2150, 2106, 2108 may be updated from time to time from an AI model 2105 present in the server 2120, where the AI model present in the server is continually trained using ultrasound frames of additional data acquired by multiple scanners 2131, 2102, 2104.

### D. Interpretation of Terms

Unless the context clearly requires otherwise, throughout the description and the
“comprise”, “comprising”, and the like are to be construed in an inclusive sense, as opposed to an exclusive or exhaustive sense; that is to say, in the sense of “including, but not limited to”;
“connected”, “coupled”, or any variant thereof, means any connection or coupling, either direct or indirect, between two or more elements; the coupling or connection between the elements can be physical, logical, or a combination thereof;
“herein”, “above”, “below”, and words of similar import, when used to describe this specification, shall refer to this specification as a whole, and not to any particular portions of this specification;
“or”, in reference to a list of two or more items, covers all of the following interpretations of the word: any of the items in the list, all of the items in the list, and any combination of the items in the list;
the singular forms “a”, “an”, and “the” also include the meaning of any appropriate plural forms.
Unless the context clearly requires otherwise, throughout the description and the claims:

Words that indicate directions such as “vertical”, “transverse”, “horizontal”, “upward”, “downward”, “forward”, “backward”, “inward”, “outward”, “vertical”, “transverse”, “left”, “right”, “front”, “back”, “top”, “bottom”, “below”, “above”, “under”, and the like, used in this description and any accompanying claims (where present), depend on the specific orientation of the apparatus described and illustrated. The subject matter described herein may assume various alternative orientations. Accordingly, these directional terms are not strictly defined and should not be interpreted narrowly.

Embodiments of the invention may be implemented using specifically designed hardware, configurable hardware, programmable data processors configured by the provision of software (which may optionally comprise “firmware”) capable of executing on the data processors, special purpose computers or data processors that are specifically programmed, configured, or constructed to perform one or more steps in a method as explained in detail herein and/or combinations of two or more of these. Examples of specifically designed hardware arc: logic circuits, application-specific integrated circuits (“ASICs”), large scale integrated circuits (“LSIs”), very large scale integrated circuits (“VLSIs”), and the like. Examples of configurable hardware are: one or more programmable logic devices such as programmable array logic (“PALs”), programmable logic arrays (“PLAs”), and field programmable gate arrays (“FPGAs”). Examples of programmable data processors are: microprocessors, digital signal processors (“DSPs”), embedded processors, graphics processors, math co-processors, general purpose computers, server computers, cloud computers, mainframe computers, computer workstations, and the like. For example, one or more data processors in a control circuit for a device may implement methods as described herein by executing software instructions in a program memory accessible to the processors.

For example, while processes or blocks are presented in a given order herein, alternative examples may perform routines having steps, or employ systems having blocks, in a different order, and some processes or blocks may be deleted, moved, added, subdivided, combined, and/or modified to provide alternative or sub combinations. Each of these processes or blocks may be implemented in a variety of different ways. Also, while processes or blocks are at times shown as being performed in series, these processes or blocks may instead be performed in parallel or may be performed at different times.

The invention may also be provided in the form of a program product. The program product may comprise any non-transitory medium which carries a set of computer-readable instructions which, when executed by a data processor (e.g., in a controller and/or ultrasound processor in an ultrasound machine), cause the data processor to execute a method of the invention. Program products according to the invention may be in any of a wide variety of forms. The program product may comprise, for example, non-transitory media such as magnetic data storage media including floppy diskettes, hard disk drives, optical data storage media including CD ROMs, DVDs, electronic data storage media including ROMs, flash RAM, EPROMs, hardwired or preprogrammed chips (e.g., EEPROM semiconductor chips), nanotechnology memory, or the like.

The computer-readable signals on the program product may optionally be compressed or encrypted.

Where a component (e.g. a software module, processor, assembly, device, circuit, etc.) is referred to above, unless otherwise indicated, reference to that component (including a reference to a “means”) should be interpreted as including as equivalents of that component any component which performs the function of the described component (i.e., that is functionally equivalent), including components which are not structurally equivalent to the disclosed structure which performs the function in the illustrated exemplary embodiments of the invention.

Specific examples of systems, methods and apparatus have been described herein for purposes of illustration. These are only examples. The technology provided herein can be applied to systems other than the example systems described above. Many alterations, modifications, additions, omissions, and permutations are possible within the practice of this invention. This invention includes variations on described embodiments that would be apparent to the skilled addressee, including variations obtained by: replacing features, elements and/or acts with equivalent features, elements and/or acts; mixing and matching of features, elements and/or acts from different embodiments; combining features, elements and/or acts from embodiments as described herein with features, elements and/or acts of other technology; and/or omitting combining features, elements and/or acts from described embodiments.

To aid the Patent Office and any readers of any patent issued on this application in interpreting the claims appended hereto, applicant wishes to note that they do not intend any of the appended claims or claim elements to invoke 35 U.S.C. 112 (f) unless the words “means for” or “step for” are explicitly used in the particular claim.

It is therefore intended that the following appended claims and claims hereafter introduced are interpreted to include all such modifications, permutations, additions, omissions, and sub-combinations as may reasonably be inferred. The scope of the claims should not be limited by the preferred embodiments set forth in the examples but should be given the broadest interpretation consistent with the description as a whole.

What is claimed is:

1. A method for automatically predicting, validating and measuring one or more biometric measurements on an ultrasound image comprising:
acquiring an ultrasound image, from an ultrasound scanner;
deploying an AI model to execute on a computing device communicably connected to the ultrasound scanner, wherein the AI model is trained so that when the AI model is deployed, the computing device identifies a feature to be measured, in whole or part and selects a definable value of the feature;
processing, using the AI model, the ultrasound image to identify a feature, in whole or part, and to select one or more definable values of the feature on the ultrasound image, together forming an AI model output;
calculate, using the AI model output, a validity score for the one or more definable values based on a threshold;
accepting each of the one or more definable values which meet or exceed the threshold and rejecting the one or more definable values which are below the threshold;
employ the one or more definable values which meet or exceed the threshold to calculate a measurement of the feature.

2. The method of claim 1 wherein the feature to be measured is a landmark feature, a measurement of the landmark feature is linear, and one or more definable values comprise co-ordinates, and the method comprises:
generating at least one heatmap using the AI model which processes the ultrasound image, wherein the ultrasound image comprises pixels, wherein said at least one heatmap comprises a probability of finding localization of said landmark feature in the pixels; and
transforming at least one heatmap to normalized coordinates of the landmark feature using a Differentiable Spatial to Numerical Transform.

3. The method of claim 2 wherein normalized co-ordinates comprise x-y co-ordinates of a centre of mass of the at least one heatmap.

4. The method of claim 3 comprising calculating the validity score for each co-ordinate, of the normalized co-ordinates, based on the intensity of the at least one heatmap relative to x-y co-ordinates of a centre of mass, wherein when the validity score for a co-ordinate is below the threshold, the co-ordinate is rejected and wherein when the validity score for a co-ordinate is at or above the threshold, the co-ordinate is accepted; calculating the measurement using the co-ordinates which are accepted.

5. The method of claim 1 wherein the AI model comprises a contracting path and an expanding path, and wherein the contracting path comprises encoding residual blocks for encoding the ultrasound image and the expansive path comprises decoding residual blocks for decoding the ultrasound images encoded by the contracting path.

6. The method of claim 2 wherein the ultrasound image comprises a fetus, in whole or part, and the measurement is selected from the group consisting of crown-rump length, biparietal diameter, and femur length.

7. The method of claim 1 additionally comprising a step of back converting the ultrasound image to a pre-scan converted ultrasound image prior to deployment of the AI model.

8. The method of claim 4 wherein a workflow application on multi-purpose electronic device, which is communicatively coupled with the ultrasound scanner, receives the AI model output, calculates the validity score, automatically places a caliper set based on the validity score, and acquires the measurement based on the co-ordinates which are accepted.

9. The method of claim 1 wherein the AI output additionally comprises a segmentation mask of the feature, in whole or in part.

10. The method of claim 1 additionally comprising a step of displaying on a screen of the computing device the measurement of the feature.

11. The method of claim 6 additionally comprising a step of calculating gestational age of the fetus from of at least one of crown-rump length, biparietal diameter, and femur length and displaying on a screen of the computing device, at least one of a measurement crown-rump length, biparietal diameter, and femur length, and gestational age.

12. The method of claim 1 wherein the feature to be measured is a feature comprising a circumference, and one or more definable values comprise a predicted contour area and the method comprises:
generating at least one segmentation mask of the feature comprising a circumference using the AI model, which processes the ultrasound image, thereby forming a contour in the segmentation mask;
fitting an ellipse on the contour;
calculate the validity score for one or more parts of the contour, based on the degree of overlap between one or more parts of the contour and the ellipse, wherein when the validity score is below the threshold, no circumference measurement is calculated and when the validity score is at or above the threshold, the contour is accepted as a high validity segmentation and the circumference of the circumferential feature is calculated.

13. The method of claim 12 wherein the ultrasound image comprises a fetus, in whole or part, and the circumference is selected from the group consisting of head circumference and abdominal circumference.

14. The method of claim 12 wherein a workflow application on multi-purpose electronic device, which is communicatively coupled with the ultrasound scanner, receives the AI model output, calculates the validity score, automatically places a caliper set based on the validity score, and acquires the circumference based on the contour which is accepted.

15. The method of claim 13 additionally comprising a step of calculating gestational age of the fetus from of at least one of head circumference and abdominal circumference and displaying on a screen of the computing device, at least one of a measurement of head circumference and abdominal circumference, and gestational age.

16. A system comprising:
an ultrasound scanner configured to acquire a new ultrasound image frame;
a computing device communicably connected to the ultrasound scanner and configured to:
process the new ultrasound image frame against a trained AI model to identify a feature on the ultrasound image, in whole or part, and to select one or more definable values of the feature on the ultrasound image, together forming an AI model output;
calculate, using the AI model output, a validity score for the one or more definable values based on a threshold;
accept each of the one or more definable values which meet or exceed the threshold and reject the one or more definable values which are below the threshold;
employ the one or more definable values which meet or exceed the threshold to calculate a measurement of the feature.

17. The system of claim 16 wherein the feature to be measured is a landmark feature, a measurement of the landmark feature is linear, and one or more definable values comprise co-ordinates, and the computing device is additionally configured to:
generate at least one heatmap using the AI model which processes the ultrasound image, wherein the ultrasound image comprises pixels, wherein said at least one heatmap comprises a probability of finding localization of said landmark feature in the pixels; and
transform at least one heatmap to normalized coordinates of the landmark feature using a Differentiable Spatial to Numerical Transform, wherein normalized co-ordinates comprise x-y co-ordinates of a centre of mass of the at least one heatmap;
calculate the validity score for each co-ordinate, of the normalized co-ordinates, based on the intensity of the at least one heatmap relative to x-y co-ordinates of a centre of mass, wherein when the validity score for a co-ordinate is below the threshold, the co-ordinate is rejected and wherein when the validity score for a co-ordinate is at or above the threshold, the co-ordinate is accepted; and
calculate the measurement using the co-ordinates which are accepted.

18. The system of claim 16 wherein the feature to be measured is a is a feature comprising a circumference, and one or more definable values comprise a predicted contour area and the computing device is additionally configured to:
generate at least one segmentation mask of the feature comprising a circumference using the AI model, which processes the ultrasound image, thereby forming a contour in the segmentation mask;
fit an ellipse on the contour;
calculate the validity score for one or more parts of the contour, based on the degree of overlap between one or more parts of the contour and the ellipse, wherein when the validity score is below the threshold, no circumference measurement is calculated and when the validity score is at or above the threshold, the contour is accepted as a high validity segmentation and the circumference of the circumferential feature is calculated.

19. The system of claim 16 additionally comprising a screen display in communication with the computing device, for the measurement of the feature.

20. A computer-readable media storing computer-readable instructions, which, when executed by a processor cause the processor to:
process a new ultrasound image frame against a trained AI model to identify a feature on the ultrasound image, in whole or part, and to select one or more definable values of the feature on the ultrasound image, together forming an AI model output;
calculate, using the AI model output, a validity score for the one or more definable values based on a threshold;
accept each of the one or more definable values which meet or exceed the threshold and reject the one or more definable values which are below the threshold;
employ the one or more definable values which meet or exceed the threshold to calculate a measurement of the feature.
